(19) United States
(12) Patent Application Publication
(10) Pub. No.: US 2026/0207823 A1
(43) Pub. Date: Jul. 23, 2026

(54) COLLAGEN FIBER HYDROGEL COMPOSITES AND METHODS

(71) Applicants: The Johns Hopkins University, Baltimore, MD (US); LifeSprout, Inc., Baltimore, MD (US)

(72) Inventors: Hai-Quan Mao, Baltimore, MD (US); Sashank Reddy, Baltimore, MD (US); Jiayuan Kong, Baitmore, MD (US); Zhicheng Yao, Baltimore, MD (US); Russell Martin, Baltimore, MD (US)

(21) Appl. No.: 19/144,186

(22) PCT Filed: Dec. 27, 2023
(86) PCT No.: PCT/US23/86104

§ 371 Date: Jun. 27, 2025

Related U.S. Application Data
(60) Provisional application No. 63/435,403, filed on Dec. 27, 2022.

(51) Int. Cl. A61L 27/52; A61L 27/20; A61L 27/24

(52) U.S. Cl. CPC A61L 27/52; A61L 27/20; A61L 27/24; A61L 2400/06

(57) ABSTRACT

Provided herein, inter alia, are collagen-based hydrogel composite, its compositions, and use thereof for soft tissue repair, or cosmetic and reconstructive purposes.

The present application claims the benefit of priority of U.S. provisional application No. 63/435,403 filed Dec. 27, 2022, which is incorporated herein by reference in its entirety.

### FIELD

Provided herein, inter alia, are soft tissue devices including hydrogel composites, compositions, and use for fat grafting and regenerating soft tissue.

### BACKGROUND

Soft tissue defects resulting from trauma, oncologic resection, or congenital malformation have been treated using fat grafting. For example, in 2019, there were 600,000 fat grafting procedures in the face worldwide, and substantially more fat grafting procedures in other anatomical regions. The tissue regrowth may need a suitable matrix for cells to attach, migrate, proliferate, differentiation, and organize into new tissue. For example, native extracellular matrix (ECM) has been used at the repair site. However, there is no current material that can endure mechanical and structural challenges of cells during the restoration.

### SUMMARY

In one aspect, a polymer (e.g., hyaluronic acid (HA))-based form of a fiber hydrogel composite is provided, with a porous hydrogel combined with dispersed fibers to mimic the fibrous structures of the body's extracellular matrix. In preferred aspects, the composite may immediately fill voids in the body, encourages cellular infiltration, macrophage polarization to pro-healing phenotypes, promotes angiogenesis, and/or enable durable soft tissue remodeling. In preferred aspects of the disclosure, provided is a hydrogel composite including fibers (e.g., nanofibers or microfibers) that can mimic the natural extracellular matrix.

In an aspect, the disclosure provides a fiber-hydrogel composite including fibers (e.g., nanofibers or microfibers) or microfibers comprising one or more extracellular matrix proteins (ECM); a hydrogel material such as hyaluronic acid (HA); and a crosslinking agent. The hydrogel material (e.g. HA) is suitably covalently bonded to the fibers by the crosslinking agent to form a composite network.

In a particular aspect, the disclosure provides a fiber-hydrogel composite including fibers (e.g., nanofibers or microfibers) or microfibers comprising one or more extracellular matrix proteins (ECM); a hydrogel material such as hyaluronic acid (HA); and a crosslinking agent. The hydrogel material (e.g. HA) is bonded to the fibers by the crosslinking agent to form a composite network.

Other suitable materials that can be utilized as a hydrogel material in the present composite and compositions include materials that can be formed into a gel, with one or preferably multiple functional groups (e.g. hydroxyl groups, amine groups, and thiol groups) that can react with a crosslinker. Hydrogel materials that have hydroxyl groups available for reaction with a crosslinker may be preferred in various aspects.

Exemplary suitable and preferred hydrogel materials include hyaluronic acid (HA), collagen, chitosan, alginate, polyvinyl acetate (PVA), gelatin, polyethylene glycol (PEG) and other glycol ethers, chondroitin sulfate or cellulose materials.

In certain aspects, a hydrogel component of the present compositions (fiber-hydrogel composite) comprises one or more of hyaluronic acid (HA), collagen, chitosan, alginate, polyvinyl acetate (PVA), gelatin, polyethylene glycol (PEG) and other glycol ethers, cellulose material. In certain aspects, a hydrogel component of the present compositions comprises hyaluronic acid (HA) and one or more of collagen, chitosan, alginate, polyvinyl acetate (PVA), gelatin, polyethylene glycol (PEG) and other glycol ethers, chondroitin sulfate or cellulose materials. In certain aspects, a hydrogel component of the present compositions comprises one or more of hyaluronic acid (HA), chitosan, alginate, polyvinyl acetate (PVA), gelatin, polyethylene glycol (PEG) and other glycol ethers, chondroitin sulfate or cellulose materials. In certain aspects, a hydrogel component of the present compositions comprises hyaluronic acid (HA) and one or more of chitosan, alginate, polyvinyl acetate (PVA), gelatin, polyethylene glycol (PEG) and other glycol ethers, chondroitin sulfate or cellulose materials. In certain aspects, a hydrogel component of the present compositions comprises, consists essentially of or consists of one or more hyaluronic acid (HA) materials.

In suitable aspects, the hydrogel component and the collagen fiber component are distinct materials, i.e. the hydrogel component and the collagen fiber component will differ in composition, molecular weight of other difference. In certain aspect, the hydrogel material will not include a collagen material.

In a particular aspect, the composition or composite comprise one or more collagen materials. In an aspect, a fiber-hydrogel composite is provided that includes fibers (e.g., nanofibers or microfibers) or microfibers comprising 1) one or more collagen materials, 2) one or more hydrogel materials such as hyaluronic acid (HA) or other material; and 3) a crosslinking agent. In preferred aspects, the hydrogen material (e.g. HA) is bonded to the fibers by the crosslinking agent to form a composite network.

In certain embodiments, the collagen of the present composite or composition (fiber hydrogel composite) comprises recombinant collagen, human collagen, and/or recombinant human collagen, respectively. In various aspects, the collagen is selected from the group consisting of collagen type I, type II, type III, type V, type V, type VI, type VII, type VIII, type IX, type X, type XI, type XII, type XIII, type XIV, type XV, type XVI, type XVII, type XVIII, type XIX, type XX, type XXI, type XXI, type XXIII, type XXIV, type XXV, type XXVI, and type XXVII. In some aspects, the collagen is collagen of one collage type free of any other collagen type; in other aspects, the collagen is a specified or unspecified mixture of more than one collagen type.

In aspects, a collagen material is present in a composition (fiber hydrogel composite) in an amount of at least about 2 mg/mL or 0.2 wt % to 200 mg/mL or of at least about or 20 wt % of one or more collagen materials based on total weight of the composite of composition at the time of gelation. In aspects, suitable composites or compositions may contain from about 1 mg/mL or 0.1 wt. % to about 150 wt. % of one or more collagen materials based on total weight of the composite of composition in the finished gel after manufacturing.

We also have found that compositions can gel more effectively if hyaluronic acid amounts (or other hydrogel materials) are above certain levels in a composition. Thus, in certain preferred compositions, hyaluronic acid is present at the time of gelation in an amount of greater than 1 wt % based on total weight of the compositions, including HA amounts of up to or at least 1.1, 1.2, 1.3, 1.4, 1.5, 1.6, 1.7, 1.8, 1.9, 2.0, 2.2, 2.4, 2.5, 2.6, 2,8, 3. 3.2, 3.4, 3.6, 3.8, 4.0, 5.0. 6.0, 7.0. 8.0. 9.0, 10.0, 11.0, or 12.0 wt. % based on total weight of the composition at the time of gelation, or equivalent concentrations in the final gel after manufacturing with suitable swelling during manufacturing of 0 (no change) to 10-fold (and thus reducing in concentration to 1/10^{th} of the gelation concentration).

We also have found that different crosslinker agents can provide enhanced gelation effects at differing amounts. Thus, with vinyl sulfone agents (e.g divinyl sulfone (DVS)), a robust gel can be produced with a HA concentration at the time of gelation of down to about 2.0% based on total weight of the composition. With a glycidyl ether (e.g. 1,4-butanediol diglycidyl ether (BDDE)) crosslinker, greater amounts of the hydrogel material such as HA can be required at the time of gelation to provide effective gelation, such as at least about 8, 10 or 12 wt % HA based on total weight of the composition.

In an aspect, the disclosure provides a soft tissue device including a fiber-hydrogel composite as described herein.

In an aspect, the disclosure provides an implant for promoting angiogenesis including a fiber-hydrogel composite as described herein.

In an aspect, the disclosure provides an implant for adipose tissue formation including a fiber-hydrogel composite as described herein.

In an aspect, the disclosure provides an implant for vasculature formation including fiber—a hydrogel composite as described herein.

In an aspect, the disclosure provides a kit including a fiber-hydrogel composite as described herein.

The disclosure can also provide simpler processes of manufacturing the fiber-hydrogel composite, which does not require a spacer within the crosslinking agent. With enhanced thermal stability in both hydrogel and fiber components, the process may include autoclaving, which enables terminal sterilization of product, an improvement in cost, risk, regulatory burden. The high thermal stability of the gel of this invention also results in shelf stability at ambient temperatures. In aspects, the composition is effective for therapeutic targets.

In an aspect, the disclosure provides a method of producing the fiber-hydrogel composite as described herein. The method includes steps of contacting a crosslinking agent with fibers comprising one or more extracellular matrix proteins (ECMs) and one or more hydrogel materials such as hyaluronic acid (HA) to obtain a fiber-hydrogel composite, wherein the one or more hydrogel materials such as HA is bonded to the fibers and to itself by the crosslinking agent to form a composite network.

In an aspect, the disclosure provides a method of forming adipose tissue formation in a subject comprising administering the fiber-hydrogel composite as described herein to the subject.

In an aspect, the disclosure provides a method of delivering a cell or tissue in a subject including encapsulating one or more cells or tissues in the fiber-hydrogel composite as described herein to form a suspension; and applying the suspension to a target site in the subject.

In an aspect, the disclosure provides a method of delivering adipose tissue in a subject including encapsulating one or more adipose tissues and the fiber-hydrogel composite as described herein to form a suspension; and applying the suspension to a target site in the subject.

In an aspect, the disclosure provides a method of delivering a pharmaceutical agent in a subject including combining a pharmaceutical agent and the fiber-hydrogel composite as described herein to form a mixture; and applying the mixture to an intended site of delivery.

In an aspect, the disclosure provides a dermal filler including the fiber-hydrogel composite as described herein.

In an aspect, the disclosure provides a soft tissue device including the fiber-hydrogel composite as described herein.

In an aspect, the disclosure provides an implant including the fiber-hydrogel composite as described herein.

In an aspect, the disclosure provides a kit including the fiber-hydrogel composite as described herein; and an applicator.

As understood, the terms fiber hydrogel composite, composition or present composition and composite are used interchangeably.

Other aspects of the invention are disclosed infra.

Where applicable or not specifically disclaimed, any one of the embodiments described herein are contemplated to be able to combine with any other one or more embodiments, even though the embodiments are described under different aspects of the invention.

### BRIEF DESCRIPTION OF THE DRAWINGS

FIG. 1A depicts a schematic for the preparation of an example of fiber-hydrogel composite by crosslinking hyaluronic acid (HA) and collagen nanofibers (electrospun and pre-crosslinked) with divinyl sulfone (DVS) or 1,4-butanediol diglycidyl ether (BDDE), generating a nanofiber-hydrogel composite (NHC).

FIG. 1B depicts morphological differences of the storage modulus (G′) equal to 100 Pascals (Pa) in hyaluronic acid (HA) and G′ equal to 250-Pa in NHC (G₀′=100-Pa).

FIG. 1C depicts a scanning electron microscope (SEM) micrograph of electrospun bovine type I collagen nanofibers with a mean diameter of approximately 600 nm.

FIG. 1D depicts that the NHC can be injected through a 27-gauge needle.

FIG. 1E depicts Scanning electron microscope (SEM) micrograph of collagen-fiber hyaluronic acid hydrogel composites showing fibral structures in close association with the hydrogel phase and a porous structure. Scale bar is 50 microns.

FIG. 2A depicts the crosslinking kinetics of HA hydrogels with 3.52 w/v % DVS at pH 12.4, 12.7, 13.0 and 13.3 prepared by different NaOH concentrations.

FIG. 2B depicts the crosslinking kinetics of HA hydrogels at pH 12.7 with different DVS concentrations (2.34 w/v %, 3.52 w/v %, and 4.68 w/v %).

FIG. 2C depicts HA hydrogels and NHCs with different DVS crosslinker concentrations (2.34 w/v % and 3.52 w/v %), and fiber amounts (0, 1, 3 w/v %) with or without interfacial bonding. The reaction pH was maintained at pH 12.7 (n=3 to 9). Statistical significance was calculated by two-way ANOVA with the Dunnett's post hoc test. Asterisk indicates the comparison between each material groups. ns, P>0.05, * P<0.05, ** P<0.01, *** P<0.001, **** P<0.0001. Data are presented as means±SEM.

FIG. 2D depicts HA hydrogels and NHCs with different fiber lengths screened by different sizes of cell strainers (40 μm, 100 μm, and unfiltered).

FIG. 2E depicts the G′ range of HA hydrogels and NHCs at different crosslinker concentrations.

FIG. 2F depicts G′ of different sets of HA hydrogel and NHC before and after particularization.

FIG. 2G depicts G′ of different sets of HA hydrogel and NHC before and after autoclaving.

FIGS. 3A-3D depict enhanced the adhesion of human adipose-derived stem cells (hADSCs) and their migration both in 2D and 3D in NHC. Cell morphology was visualized by staining for F-actin with Alexa Fluor 568 Phalloidin. Cell nuclei were stained with 4′,6-diamidino-2-phenylindole (DAPI). Statistical significance was calculated by two-way ANOVA with the Dunnett's post hoc test. Asterisk indicates the comparison between each material groups. ns, P>0.05, * P<0.05, ** P<0.01, *** P<0.001, **** P<0.0001. Data are presented as means±SEM.

FIG. 3A depicts the result of live/dead staining showing percentage of live hADSCs in the 250-Pa NHC and 100- and 250-Pa HA hydrogel controls at day 1, 4 and 7 (n=3 to 4).

FIG. 3B depicts the percentage of adhered cells in HA hydrogels and NHC using hADSCs to show the cell adhesion after culturing for 4, 24 and 48 hours (n=4).

FIG. 3C depicts enhanced cell migration and spreading inside 250-Pa NHC using hADSC cell spheroids at day 7. Scale bars, 50 μm.

FIG. 3D depicts cell spreading and migration of hADSCs on fiber mats not seen in the fiber-free hydrogel controls.

FIG. 4A depicts the macroscopic image of the NHC gels during sample collection, demonstrating retention of the NHCs in vivo at postoperative day (POD) 7, 14, 56, and 180 (scale bar=1 cm).

FIG. 4B depicts quantitative measurements of the retained shapes of 100- and 250-Pa HA hydrogels and 250-Pa NHC using calipers at POD 7, 14, 56, and 180 with initial injection of 200 μL (n=3).

FIG. 4C depicts enhanced host cell infiltration in 250-Pa NHC in vivo at POD 7. Scale bars, 500 μm.

FIG. 4D depicts signs of neo-tissue, blood vessel and adipose tissue formation in 250-Pa NHC at POD 56. Scale bars, top images=500 μm; bottom images=100 μm.

FIG. 4E depicts quantitative analysis of the infiltration area, defined by the percentage area that is infiltrated by host cells inside the entire injected matrix (n=3).

FIG. 4F depicts quantitative analysis of the density of the host cells (n=3).

FIG. 5A depicts tile-scans immunofluorescence images to show enhanced CD68+ pan-macrophage (red), CD38+ M1 macrophage and CD163+ M2 macrophage infiltration and expression at POD 7. Scale bars, tile-scans=200 μm.

FIG. 5B depicts zoomed in Z-stack immunofluorescence images to show enhanced CD68+ pan-macrophage (red), CD38+ M1 macrophage and CD163+ M2 macrophage infiltration and expression at POD 7. Scale bars, Z-stack images=50 μm.

FIGS. 5C-5D depict relatively low CD38+ M1 macrophage expression (FIG. 5C) and high CD163+ M2 macrophage expression (FIG. 5D) in 250-Pa NHC at POD 56. Scale bars, 200 μm.

FIGS. 5E-5H depict a quantitative representation of infiltrated CD68+ pan-macrophages (FIG. 5E), CD38+M1 macrophages (FIG. 5F), CD163+M2 macrophages (FIG. 5G) and the ratio of M2 to M1 macrophages (FIG. 5H) by analyzing the tile-scan images (n=3) at POD 56. Statistical significance was calculated by two-way ANOVA with the Dunnett's post hoc test. Asterisk indicates the comparison between each material groups. ns P>0.05, * P<0.05, ** P<0.01, *** P<0.001, **** P<0.0001. Data are presented as means±SEM.

FIG. 5I depict graphs of gene expression level of tested genes separated by category: macrophages (CD68, Nos2, CD86, Arg1, CD163, CD38), inflammation (TNF-α, IL1b), and anti-inflammation (TGF-β, IL10, IL13). Expression is normalized to d7 250-Pa HA. Samples were pooled together then tested in triplicate, with n=3. Statistical significance was calculated by two-way ANOVA with the Dunnett's post hoc test. Asterisk indicates the comparison between each material groups. ns P>0.05, * P<0.05, ** P<0.01, *** P<0.001, **** P<0.0001. Data are presented as means±SEM. FIG. 6A depicts host endothelial cell and smooth muscle in-growth in the injected 100- and 250-Pa HA hydrogels and the 250-Pa NHC at POD 7, 14, and 56. Endothelial cells were stained with RECA-1, smooth muscles were stained with α-SMA (green) and all infiltrated cell nuclei were stained with DAPI. Scale bars, 200 μm for tile-scans (left); 50 μm for Z-stack images (right).

FIG. 6B depicts quantitative analysis of the endothelial cell infiltration area inside the hydrogels and the composite (n=6).

FIG. 6C depicts quantitative analysis of the endothelial cell infiltration rate inside the hydrogels and the composite (n=6).

FIG. 6D depicts quantitative analysis of the blood vessel density inside the hydrogels and the composite (n=6).

FIG. 6E depicts gene expression level of tested genes for angiogenesis (CD31 and VEGF-a). Expression is normalized to d7 250-Pa HA. Samples were pooled together then tested in triplicate, with n=3. Statistical significance was calculated by two-way ANOVA with the Dunnett's post hoc test. Hash key (#) denotes the comparison between the 100-Pa HA hydrogel and the 250-Pa NHC. Asterisk (*) indicates the comparison between the 250-Pa HA control and the 100-Pa HA or the 250-Pa NHC. ns P>0.05, #or *P<0.05, ##or **P<0.01, ###or ***P<0.001, ####or ****P<0.0001. Data are presented as means±SEM FIG. 7A depicts NHC-mediated adipocyte and adipose tissue formation. FIG. 7A depicts H&E staining images of 100- and 250-Pa HA and 250-Pa NHC gels on Day 180. Scale bar=100 μm.

FIG. 7B depicts high and low magnification H&E staining images of 250-Pa NHC gel on Day 180. Scale bar=5 mm for low magnification, top image and 100 μm for high magnification images. Arrows in left high magnification image indicate blood vessels, and arrows in right high magnification image indicate the interface between the NHC and adipocytes.

FIG. 7C depicts Immunocytochemistry images for 250-Pa NHC on Day 14, 56 and 180. Adipocytes were stained with Perilipin-1 (green) and all infiltrated cell nuclei were stained with DAPI (blue). Scale bars=200 μm.

FIG. 7D depicts improved adipocyte and adipose tissue formation in the injected 250-Pa NHC at POD 14, and 56 relative to 100 and 250 Pa gels without fibers. Adipocytes were stained with Acrp-30, adipose tissues were stained with Perilipin-1 and all infiltrated cell nuclei were stained with DAPI (blue). Scale bars, 200 μm for tile-scans (left); 50 μm for Z-stack images (right).

FIG. 7E depicts close localization of Perilipin-1+ adipose tissue formation (green) with RECA-1+ endothelial cells (red) at POD 14 (left column) and 56 (right column). Scale bars, 200 μm.

FIG. 7F depicts infiltration of CD107a+ perivascular progenitor cells (gray) and its close localization with RECA-1+ endothelial cells (green) and Acrp-30+ adipocytes (red) at POD 14 inside NHC. Scale bar, 200 μm.

FIG. 7G depicts quantitative analysis of the density of adipose tissues inside the NHC and HA hydrogels (n=3) by Acrp30+ staining.

FIG. 7H depicts quantitative analysis of the area converted to adipocytes within the NHC and HA hydrogels at day 180 (n=3).

FIG. 7I depicts quantitative analysis of the density of adipocytes inside the NHC and HA hydrogels (n=3) by perilipin staining at days 14, 56, and 180.

FIG. 7J depicts quantitative analysis of the diameter of adipocytes inside the NHC and HA hydrogels (n=3) by perilipin staining at days 14, 56, and 180. Statistical significance was calculated by one-way ANOVA with the Dunnett's post hoc test to compare between groups. ***P<0.001, ****P<0.0001. Data are presented as means±SEM.

FIG. 8 depicts the HA concentration requirement to form robust gels.

FIG. 9A depicts the live/dead cell viability percentage of HUVECs in the 250-Pa NHC and 100- and 250-Pa HA hydrogel controls at day 1, 4 and 7 (n=3 to 4).

FIG. 9B depicts the percentage of adhered cells in HA hydrogels and NHC using HUVECs to show the cell adhesion after culturing for 4, 24 and 48 hours (n=4).

FIG. 9C depicts the close localization of CD163+ M2-like pro-regenerative macrophages with host endothelial cells inside 250-Pa NHC at POD 14.

FIG. 9D depicts infiltration of CD107a+ progenitor cells (gray) and its correlation with RECA-1+ endothelial cells and Acrp-30+ adipocytes at POD 14 inside (A) 100-Pa HA, (B) 250-Pa HA and (C-D) 250-Pa NHC. Scale bar, 200 rm.

FIG. 9E depicts immunocytochemistry images for 250-Pa NHC on Day 14, 56 and 180 to show the close localization of adipocytes (stained with perilipin-1) with blood vessels (stained with RECA-1),

FIG. 9F depicts immunocytochemistry images for 250-Pa NHC on Day 180 to show the close localization of blood vessels (RECA-1 positive) with pre-adipocytes (Pref-1 positive).

FIG. 9G depicts immunocytochemistry images for 250-Pa NHC on Day 14, 56 and 180 to show the close localization of endothelial cells (stained with RECA-1) with perivascular cells (stained with PDGFRα+)

FIG. 9H depicts the graphs of quantitative analysis of the density of PDGFRα+ perivascular cells (left) and pre-adipocytes (right) on Day 14, 56 and 180 in 250-Pa NHC. Statistical significance was calculated by one-way ANOVA with the Dunnett's post hoc test to compare between groups. ***P<0.001, ****P<0.0001. Data are presented as means±SEM.

FIG. 10 depicts collagen-fibers electrospun from porcine type 1 atelocollagen (Nitta) suitable for preparation of fiber-hydrogel composites.

FIG. 11 depicts collagen-fibers electrospun from porcine gelatin (Sigma) suitable for preparation of fiber-hydrogel composites. The fibers formed as ribbons, demonstrating additional morphological options.

FIG. 12 depicts collagen-fibers electrospun from recombinant collagen (Vecollan from Evonik) suitable for preparation of fiber-hydrogel composites.

FIG. 13 depicts optical microscope micrograph of electrospun recombinant collagen (Vecollan by Evonik) after crosslinking and stained with picro Sirius red dye. After crosslinking, it is stable in aqueous environments and can be formed into composites with Hyaluronic acid and retain fibrous morphology after gelation and autoclave sterilization.

FIG. 14 depicts collagen-fibers electrospun from recombinant collagen (Demulcent SFA by Jland) suitable for preparation of fiber-hydrogel composites.

FIG. 15 depicts optical microscope micrograph of electrospun recombinant collagen (Demulcent SFA by Jland) after crosslinking and stained with picro Sirius red dye. After crosslinking, it is stable in aqueous environments and can be formed into composites with Hyaluronic acid and retain fibrous morphology after gelation and autoclave sterilization.

### DETAILED DESCRIPTION

The following detailed description, given by way of example, but not intended to limit the invention solely to the specific embodiments described, may best be understood in conjunction with the accompanying drawings.

In one aspect, pre-reacted, beaded composite materials are provided comprising a hydrogel and a nanostructure for use in methods for reconstruction of soft tissue. The invention also relates to a soft tissue device comprising beaded composite materials for cell and tissue delivery for cosmetic, reconstructive, and cellular therapies.

In particular preferred aspects, composite materials are provided that can recruit, capture, encapsulate, associate, and/or embed specific tissue constituents including but not limited to adipocytes, other mesenchymal cells, or mesenchymal stem cells. In preferred aspects, composite materials are provided that can recruit, capture, encapsulate, associate, and/or embed specific tissues including but not limited to adipose tissues.

In further preferred aspects, methods are provided for repairing or reconstructing a soft tissue injury using a composition comprising a scaffold complex (such as soft tissue device) comprising a biomaterial covalently linked to a biodegradable fiber, such as collagen.

In additional aspects, methods of fabricating a composition for use in soft tissue reconstruction are provided where the composition comprises a hydrogel and a nanostructure disposed therein. The invention in particular aspects also relates to a method of fabricating a composition for use in cell and tissue delivery for cosmetic, reconstructive, and cellular therapies.

The following is a detailed description of the invention provided to aid those skilled in the art in practicing the present invention. Those of ordinary skill in the art may make modifications and variations in the embodiments described herein without departing from the spirit or scope of the present invention. Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. All publications, patent applications, patents, figures and other references mentioned herein are expressly incorporated by reference in their entirety.

Although any methods and materials similar or equivalent to those described herein can also be used in the practice or testing of the present invention, the preferred methods and materials are now described. All publications mentioned herein are incorporated herein by reference to disclose and described the methods and/or materials in connection with which the publications are cited.

Unless defined otherwise, all technical and scientific terms used herein have the meaning commonly understood by a person skilled in the art to which this invention belongs. The following references, the entire disclosures of which are incorporated herein by reference, provide one of skill with a general definition of many of the terms (unless defined otherwise herein) used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, the Harper Collins Dictionary of Biology (1991). Generally, the procedures of molecular biology methods described or inherent herein and the like are common methods used in the art. Such standard techniques can be found in reference manuals such as for example Sambrook et al., (2000, Molecular Cloning—A Laboratory Manual, Third Edition, Cold Spring Harbor Laboratories); and Ausubel et al., (1994, Current Protocols in Molecular Biology, John Wiley & Sons, New-York).

The following terms may have meanings ascribed to them below, unless specified otherwise. However, it should be understood that other meanings that are known or understood by those having ordinary skill in the art are also possible, and within the scope of the present invention. All publications, patent applications, patents, and other references mentioned herein are incorporated by reference in their entirety. In the case of conflict, the present specification, including definitions, will control. In addition, the materials, methods, and examples are illustrative only and not intended to be limiting.

### Definitions

The term “a” and “an” refers to one or to more than one (i.e., to at least one) of the grammatical object of the article. By way of example, “an element” means one element or more than one element.

As used herein, “about” can mean plus or minus less than 1 or 1, 2, 3, 4, 5, 6, 7, 8, 9, 10, 11, 12, 13, 14, 15, 16, 17, 18, 19, 20, 25, 30, or greater than 30 percent, depending upon the situation and known or knowable by one skilled in the art.

As used herein the specification, “subject” or “subjects” or “individuals” may include, but are not limited to, mammals such as humans or non-human mammals, e.g., domesticated, agricultural or wild, animals, as well as birds, and aquatic animals. In certain embodiments, the subject is a human patient or an animal subjected to medical treatment.

As used herein, the term “hydrogel” is a type of “gel,” and refers to a water-swellable polymeric matrix, consisting of a three-dimensional network of macromolecules (e.g., hydrophilic polymers, hydrophobic polymers, blends thereof) held together by covalent or non-covalent crosslinks that can absorb a substantial amount of water (e.g., 50%, 60% 70%, 80%, 90%, 95%, 96%, 97%, 98%, 99% or greater than 99% per unit of non-water molecule) to form an elastic gel. The hydrogel may contain “water-swellable” polymer is one that absorbs an amount of water greater than at least 50% of its own weight, upon immersion in an aqueous medium. The polymeric matrix may be formed of any suitable synthetic or naturally occurring polymer material. As used herein, the term “gel” refers to a solid three-dimensional network that spans the volume of a liquid medium and ensnares it through surface tension effects. This internal network structure may result from physical bonds (physical gels) or chemical bonds (chemical gels), as well as crystallites or other junctions that remain intact within the extending fluid. Virtually any fluid can be used as an extender including water (hydrogels), oil, and air (aerogel). Both by weight and volume, gels are mostly fluid in composition and thus exhibit densities similar to those of their constituent liquids. A hydrogel is a type of gel that uses water as a liquid medium.

In certain embodiments, the hydrogel is a composite or composite material. The term “composite” as used herein includes any association, bonding or attachments of two or more components. In some embodiments, the “hydrogel composite” as used herein include at least a polymeric fiber and a hydrogel material. The hydrogel composite contains the polymeric fiber (e.g., collagen, gelatin, etc) and hydrogel material (e.g., hyaluronic acid (HA)).

A term “functional network” as used herein means that the interactions between components results in a chemical, biochemical, biophysical, physical, or physiological benefit. In addition, a functional network may include additional components, including cells, biological materials (e.g., polypeptides, nucleic acids, lipids, carbohydrates), therapeutic compounds, synthetic molecules, and the like. In certain embodiments, the scaffold complex promotes tissue growth and cell infiltration when implanted into a target tissue present in a human subject.

The term “nanofiber” or “microfiber” can be used interchangeably to refer to fibers that are thousands of nanometers in diameter, such as 1 micron to 10 microns.

The term “nanofiber” as used herein refers to a fibrous material having at least one dimension (e.g., length, or width) less than about 999 nm, less than about 900 nm, less than about 800 nm, less than about 700 nm, less than about 600 nm, less than about 500 nm, less than about 400 nm, less than about 300 nm, less than about 200 nm, less than about 100 nm, less than about 50 nm, or less than about 10 nm. In some embodiments, the nanofibers may have a length less than about 999 nm, less than about 900 nm, less than about 800 nm, less than about 700 nm, less than about 600 nm, less than about 500 nm, less than about 400 nm, less than about 300 nm, less than about 200 nm, less than about 100 nm, less than about 50 nm, or less than about 10 nm. In some embodiments, the nanofibers may have a width less than about 999 nm, less than about 900 nm, less than about 800 nm, less than about 700 nm, less than about 600 nm, less than about 500 nm, less than about 400 nm, less than about 300 nm, less than about 200 nm, or less than about 100 nm.

The term “microfiber” as used herein refers to a fibrous material having at least one dimension (e.g., length, or width) less than about 100 μm, less than about 90 μm, less than about 80 μm, less than about 70 μm, less than about 60 μm, less than about 50 μm, less than about 40 μm, less than about 30 μm, less than about 20 μm, or less than about 10 μm. In some embodiments, the microfibers may have a length than about 10 μm, less than about 8 μm, less than about 7 μm, less than about 6 μm, less than about 5 μm, less than about 4 μm, less than about 3 μm, less than about 2 μm, or less than about 1 μm. In certain embodiments, the microfibers may have a length between about 1 μm and 10 μm, between about 5 μm and 10 μm, or between about 8 μm and 10 μm. In certain embodiments, the microfibers may have a length about 8 μm or 10 μm.

The term “fiber-hydrogel composite” as used herein refers to a composite including at least fibers such as nanofibers, microfibers, or combination thereof (e.g., polymeric fibers, or nanofibers made of extracellular matrix proteins) and hydrogel component (e.g., HA), which form functional networks. In addition, the “nanofiber-hydrogel composite,” “fiber-hydrogel composite,” “hydrogel composite,” “composite” or “complex” as used herein are interchangeably used referring to such composite including at least fibers (e.g., collagen or gelatin nanofibers) and hydrogel component (e.g., HA).

The term “crosslinked” herein refers to a composition containing intramolecular and/or intermolecular crosslinks, whether arising through covalent or noncovalent bonding, and may be direct or include a cross-linker. “Noncovalent” bonding includes both hydrogen bonding and electrostatic (ionic) bonding.

The term “polymer” includes linear and branched polymer structures, and also encompasses crosslinked polymers as well as copolymers (which may or may not be crosslinked), thus including block copolymers, alternating copolymers, random copolymers, and the like. Those compounds referred to herein as “oligomers” are polymers having a molecular weight below about 1000 Da, preferably below about 800 Da. Polymers and oligomers may be naturally occurring or obtained from synthetic sources.

The term “extracellular matrix protein” (“ECM”) as used herein refers to a protein or its macromolecular matrix including a protein, which mimics a three-dimensional network protein existing outside of a cell (e.g., animal or mammalian cell or plant cell) and providing structural and biochemical support or adhesion to surrounding the cell. Exemplary ECM may include collagen, gelatin, elastin, decellularized matrix, or derivatives thereof.

As used herein, the term “biodegradable” refers to a material that can be broken down by biological means in a subject.

As used herein, the term “implantable” or “injectable” means able to be formulated for implantation into or on a subject, such as via a syringe or a device to a subject. For example, the implantable means may include syringe delivery, versus mesh, paste, or the like.

As used herein, the term “soft tissue” refers to tissues that connect, support, or surround other structures and organs of the body. Soft tissue includes muscles, tendons, ligaments, fascia, nerves, fibrous tissues, fat, blood vessels, and synovial membranes.

As used herein, the term “stable” refers to a material property that does not degrade significantly at a given condition (such as room temperature) over a given timeframe. In certain embodiments, the “stable” refers to a material property that does not degrade significantly at a certain temperature, or after treatment at a certain temperature. For example, if the material is stable during autoclave, it is meant that the material does not degrade significantly during the process of autoclaving (e.g., steaming), or at the range of temperature during the process of autoclaving (e.g., steaming).

As used herein, the term “autologous” refers to any material derived from the same individual to whom it is later to be re-introduced into the individual.

As used herein, the term “allogeneic” or, alternatively, “allogenic,” refers to any material derived from a different animal of the same species or different patient as the individual to whom the material is introduced.

As used herein, the term “functionalized” refers to a material that is uniformly or non-uniformly modified so as to have a functional chemical moiety associated therewith (e.g., chemically modified). In some cases, functional chemical moiety is capable of reacting to permit the formation of a covalent or non-covalent bond. In some cases, functional chemical moiety can provide the material improved properties.

### Fiber-Hydrogel Composite

Provided is a “fiber-hydrogel composite,” “hydrogel composite,” or “composite” that is formed by combining hydrogel materials or other biomaterials (e.g., cells, macrophages, cell digest or cell debris) with fibers (e.g., nanofibers or microfibers) including those with diameters in the range of 1 nm to 10 microns.

In particular, the fibers (e.g., nanofibers or microfibers) suitably include one or more extracellular matrix proteins (ECM).

In particular, the fibers (e.g., nanofibers or microfibers) include one or more collagen materials. In a preferred aspect, the collagen materials may comprise a recombinant collagen material, including a human recombinant collagen material.

In one aspect, a fiber-hydrogel composite includes (i) fibers (e.g., nanofibers or microfibers) comprising one or more extracellular matrix proteins (ECM), and particularly one or more collagen materials; (ii) a hyaluronic acid (HA); and (iii) a crosslinking agent. The HA is bonded (e.g., covalently or non-covalently) to the fibers by the crosslinking agent to form a composite network.

In certain embodiments, the fibers (e.g., nanofibers or microfibers) have a mean diameter of less than about 10 μm, less than about 5 μm, less than about 4 μm, less than about 3 μm, less than about 2 μm, less than about 1 μm, less than about 900 nm, less than about 800 nm, less than about 700 nm, less than about 600 nm, less than about 500 nm, less than about 400 nm, less than about 300 nm, less than about 200 nm, less than about 100 nm, less than about 50 nm, less than about 10 nm, less than about 5 nm, or less than about 1 nm. In certain embodiments, the fibers have a mean diameter in a range of about 10 nm to 5 μm, about 100 nm to 5 μm. In certain embodiments, the fibers have a mean diameter in a range of about 1 nm to 1,000 nm, about 1 nm to 500 nm, or about 1 nm to 100 nm.

In certain embodiments, the fibers (e.g., microfibers) have a length greater than about 1 μm, greater than about 5 μm, greater than about 10 μm, greater than about 20 μm, greater than about 30 μm, greater than about 40 μm, greater than about 50 μm, greater than about 60 μm, greater than about 70 μm, greater than about 80 μm, greater than about 90 μm, greater than about 100 μm, greater than about 200 μm, greater than about 300 μm, greater than about 400 μm, greater than about 500 μm, greater than about 600 μm, greater than about 700 μm, greater than about 800 μm, greater than about 900 μm, or of about 1 mm. In certain embodiments, the fibers (e.g., microfibers) have a length in the range of about 1 to 1,000 μm, of about 10 to 500 μm, or of about 100 to 500 μm. The diameter and length of the fibers (e.g., nanofibers or microfibers) may be determined using optical (including fluorescence) microscopy or electron microscopy.

In certain embodiments, the fibers can be cylindrical (with a similar cross-section width and height) or ribboned in shape (with the cross-section width greater than the height).

Preferably, the fibers (e.g., nanofibers or microfibers) may have an aspect ratio in a range of at least about 10 to about at least 10,000. It will be appreciated that, because of the very small diameter of the fibers, the fibers have a high surface area per unit of mass. This high surface area to mass ratio permits fiber-forming solutions or liquids to be transformed from liquid or solvated fiber-forming materials to solid fibers in fractions of a second.

In certain embodiments, the fibers (e.g., nanofibers or microfibers) are functionalized. In certain embodiments, the fibers (e.g., nanofibers or microfibers) are functionalized with groups comprising hydroxyl, amino, carboxyl, thio, acrylate, sulfonate, phosphate, maleimide, amide, as well as modified forms thereof, such as activated or protected forms. In certain embodiments, any functional groups that can be reacted with a crosslinker, especially the epoxy and vinyl sulfonyl groups of a crosslinker, may be used. Examples of functional groups that are reactive to the epoxy or vinyl sulfonyl groups of a crosslinker include hydroxyl, carboxyl, thiol, or amino groups.

The nanofibers may include, but not limited to, nanofibers, nanotubes, nanofilaments, mesh sections, branched filaments or networks. The nanofibers may also comprise any suitable chemical functional groups to facilitate the covalent or noncovalent crosslinking between the nanofibers and the polymers of the hydrogels of the invention. Method, techniques, and materials are well known in the art for making and functionalizing nanofibers. Any microfabrication methods are used to make the nanofibers. In various embodiments, the disclosed devices can be assembled and/or manufactured using any suitable microfabrication technique. Such methods and techniques are widely known in the art.

Particularly, the fibers (e.g., nanofibers or microfibers) in the fiber-hydrogel composite include one or more extracellular matrix proteins (ECMs). In certain embodiments, the fibers (e.g., nanofibers or microfibers) suitably include one or more selected from collagen, gelatin, elastin, elastin-like polypeptides, tropoelastin, decellularized matrix, and hyaluronic acid. In certain embodiments, the fibers (e.g., nanofibers or microfibers) include one or more from bovine type I collagen, gelatin, or derivatives. In certain embodiments, the fibers (e.g., nanofibers or microfibers) include one or more collogen including bovine type I collagen and a recombinant collagen which may be a human recombinant collagen material.

In certain embodiments, the fiber-hydrogel composite may include a natural extracellular matrix for fibers (e.g., nanofibers or microfibers). In certain embodiments, the fiber-hydrogel composite may include a synthetic extracellular matrix for fibers (e.g., nanofibers or microfibers). In certain embodiments, the one or more ECMs include a collagen nanofiber, which may be naturally obtained or synthesized. In certain embodiments, the collagen nanofiber includes a type I bovine collagen nanofiber or fragments thereof. In certain embodiments, the collagen nanofibers may be obtained from natural sources, or may be fabricated or prepared from a composition (resin composition) including collagen. For example, the collagen nanofiber may be formed by electrospinning, centrifugal spinning, blow spinning, or combinations thereof. Particularly, collagen nanofibers are preferably prepared by electrospinning.

“Human recombinant collagen” or “human collagen material” or other similar term suitably may collagen type I, type II, type III, type IV, type V, type VI, type VII, type VIII, type IX, type X, type XI, type XII, type XIII, type XIV, type XV, type XVI, type XVII, type XVIII, type XIX, type XX, type XXI, type XXII, type XXIII, type XXIV, type XXV, type XXVI, and type XXVII or the component chains thereof. The collagen can be collagen of one type free of any other-type, or can be a mixture of collagen types. Suitably, the collagen comprises, or consists essentially of, collagens selected from the group consisting of type l collagen, type III collagen, and mixtures thereof. Human recombinant collagen suitably may be manufactured eg. by culturing a non-human organism to express at least one human gene encoding a collagen. The suitable collagen may or may not include hydroxyproline residues or telopeptide sequences.

Human recombinant collagen may be provided by any suitable method known in the art, including as disclosed in U.S. Pat. Nos. 5,962,648 and 5,593,859 and WO2004/078120. Suitably collagen will be recombinantly manufactured by culturing a cell which has been transfected with at least one gene encoding the polypeptide comprising collagen and genes encoding the oc and subunits of the post-translational enzyme prolyl 4-hydroxylase and purifying the resultant collagen monomer therefrom. The recombinant collagen solution may be subsequently subjected to polymerizaiton or cross-linking conditions.

Bovine collagen suitably can be a mixture of collagen type I (85%) and collagen type III (15%). An advantage of recombinant collagen is that collagen type I and collagen type III are made independently of one another, and so any combination of type I and type III collagen can be made. The present compositions and composites may suitably comprise human collagen type I and human collagen type III in any ratio. For example, the compositions and composites may comprise human collagen type I and human collagen type III in a ratio by weight of 100:0, 80:20. 60:40, 50:50, 40:60, 20:80 or 0:100, or anywhere in-between. Preferably, the ratio by weight of human collagen type I human collagen type III is greater than about 50:50, and preferably it is greater than about 70:30, for example about 80:20. Suitably, the type I human recombinant collagen makes up at least about 75% by weight of the total human recombinant collagens in the material.

A-s discussed above, in certain aspects, the collagen of the present compositions and composites is recombinant collagen, human collagen, or recombinant human collagen, respectively. In various embodiments, the collagen is selected from the group consisting of collagen type I, type II, type III, type IV, type V, type VI, type VII, type VIII, type IX, type X, type XI type XII, type XIII, type XIV, type XV, type XVI, type XVII, type XVIII, type XIX, type XX type XXI, type XXII, type XXIII, type XXIV, type XXV, type XXVI, and type XXVII In some embodiments, the collagen is collagen of one collage type free of any other collagen type; in other embodiments, the collagen is a specified or unspecified mixture of more than one collagen type.

In one aspect, the present composites and compositions comprise collagen, wherein the composite has a surface area greater than about 2.3 nm/g collagen. In other aspects, the composite has a surface area greater than about 2.5, 2.75, 3.0, 3.25, 3.5, 3.75, 3.8, 4.0, and 4.4 m²/g collagen In a particular aspect, a composite is provided comprising collagen, wherein the composite has a surface area of or greater than about 4.0 m²/g collagen.

In further aspects, the collagen is human collagen, recombinant collagen, recombinant human collagen, and/or collagen type I, respectively. In a preferred aspect, the collagen is recombinant human type I collagen. In certain aspects the collagen is selected from the group consisting of collagen type I, type II, type III, type IV, type V, type VI, type VII, type VIII, type IX, type X, ty pe XL, type XII type XIII, type XIV, type XV, type XVI, type XVII, type XVIII, type XIX, type XX, type XXI, type XXII, type XXIII, type XXIV, type XXV, type XXVI, and type XXVII.

The fibers (e.g., nanofibers or microfibers) suitably may preferably be fabricated by electrostatic spinning (also referred to as electrospinning). The process of electrospinning generally involves the introduction of a liquid into an electric field, so that the liquid is caused to produce fibers. These fibers are generally drawn to a conductor at an attractive electrical potential for collection. During the conversion of the liquid into fibers, the fibers harden and/or dry. This hardening and/or drying may be caused by cooling of the liquid, i.e., where the liquid is normally a solid at room temperature; by evaporation of a solvent, e.g., by dehydration (physically induced hardening); or by a curing mechanism (chemically induced hardening). Electrostatically spun fibers can be produced having very thin diameters. Parameters that influence the diameter, consistency, and uniformity of the electrospun fibers include the polymeric material and cross-linker concentration (loading) in the fiber-forming combination, the applied voltage, and needle collector distance.

The electrospun fibers (e.g., collagen nanofiber) may provide superior properties, e.g., high porosity in the hydrogel phase and mechanical reinforcement from the solid fiber component, which may be beneficial for optimal cell infiltration properties and structural integrity.

In certain embodiments, collagen fibers are prepared by electrospinning the fibers in a solution or suspension containing 1,1,1,3,3,3-hexafluoro-2-propanol (HFIP) solvent. In other embodiments, the collagen fibers are prepared by electrospinning in a solution or suspension containing different solvents, such as trifluoroethanol (TFE), trifluoroacetic acid (TFA), acetic acid, ethanol, or phosphate mixtures. In certain embodiments, the suitable solvent may include at least one of 1,1,1,3,3,3 hexafluoro-2-propanol (HFIP), 2,2,2-trifluoroethanol (TFE), and a mixture of water and acetic acid. Other solvents that may be used or combined with other solvents in electrospinning natural matrix materials, such as collagen fibers, include acetamide, N-methylformamide, N,N-dimethylformamide (DMF), dimethylsulfoxide (DMSO), dimethylacetamide, N-methyl pyrrolidone (NMP), ethyl acetate, acetonitrile, trifluoroacetic anhydride, 1,1,1-trifluoroacetone, maleic acid, and hexafluoroacetone.

Selection of a solvent will depend upon the characteristics of the synthetic polymer to be electrodeposited, such as the secondary forces that stabilize polymer-polymer interactions and the solvent's ability to replace these with strong polymer-solvent interactions. In the case of polypeptides such as collagen, and in the absence of covalent cross-linking, the principal secondary forces between chains are: (1) Coulombic, resulting from attraction of fixed charges on the backbone and dictated by the primary structure (e.g., lysine and arginine residues will be positively charged at physiological pH, while aspartic or glutamic acid residues will be negatively charged); (2) dipole-dipole, resulting from interactions of permanent dipoles—the hydrogen bond, commonly found in polypeptides, is the strongest of such interactions; and (3) hydrophobic interactions, resulting from association of non-polar regions of the polypeptide due to a low tendency of non-polar species to interact favorably with polar water molecules.

Thus, solvents or solvent combinations that may favorably compete for these interactions may dissolve or disperse polypeptides. For example, HFP and TFE possess a highly polar hydroxyl group adjacent to a very hydrophobic fluorinated region. While not wishing to be bound by theory, it is believed that the alcohol portion may hydrogen bond with peptides, and may also solvate charges on the backbone, thus reducing Coulombic interactions between molecules. Additionally, the hydrophobic portions of these solvents may interact with hydrophobic domains in polypeptides, helping to resist the tendency of the latter to aggregate via hydrophobic interactions. Solvents such as HFP and TFE, due to their lower overall polarities compared to water, may not compete well for intramolecular hydrogen bonds that stabilize secondary structures such as an alpha helix. Consequently, alpha helices in these solvents are believed to be stabilized by virtue of stronger intramolecular hydrogen bonds. The stabilization of polypeptide secondary structures in these solvents is believed to be desirable, especially in the cases of collagen and elastin, to preserve the proper formation of collagen fibrils during electrospinning. In some embodiments, solvents are selected based on their tendency to induce helical structure in electrospun protein fibers, thereby predisposing monomers of collagen or other proteins to undergo polymerization and form helical polymers that mimic the native collagen fibril. Examples of such solvents include halogenated alcohols, preferably fluorinated alcohols (HFP and TFE), hexafluoroacetone, chloroalcohols in conjugation with aqueous solutions of mineral acids and dimethylacetamide, preferably containing lithium chloride. HFP and TFE are more preferred. In some embodiments, water is added to the solvents.

In certain embodiments, the collagen nanofibers are prepared using an alternate fiber stabilizer, vapor phase glutaraldehyde. Treatment with glutaraldehyde results in crosslinking of collagen fibers, as the aldehyde groups of glutaraldehyde react with the free lysine or hydroxylysine groups on collagen fibers to form Schiff base structures. For example, a six-hour vapor phase glutaraldehyde treatment results in increased tensile strength, elasticity, stretchability, and stability of collagen fibers. In certain embodiments, collagen nanofibers are prepared using alternate collagen stabilizers or crosslinkers, such as D-ribose. As disclosed in U.S. Pat. No. 4,971,954, incorporated herein in its entirety, D-ribose can crosslink collagen fibers, resulting in a non-toxic and non-immunogenic matrix.

In certain embodiments, the collagen nanofibers are prepared using DVS (divinyl sulfone) as fiber stabilizer, either alone or in sequence with another stabilizer such as vapor phase glutaraldehyde. This can result in crosslinking of collagen fibers to stabilize the physical structure of the fibers prior to reaction during gelation. The choice of fibers crosslinking conditions (including solvent choice) can be tuned to crosslink fibers, yet leave abundant functional groups for subsequent reaction to form interfacial bonding during composite gelation.

In certain embodiments, EDC and fibers (e.g., nanofibers or microfibers) may be crosslinked (e.g., via a crosslinking moiety or directly linked). For example, form of interaction of EDC and fibers (e.g., nanofibers or microfibers) may be effective to introduce bonding (e.g., covalent bonding) therebetween.

In certain embodiments, the hydrogel material such as HA may be covalently bonded to the fibers (e.g., nanofibers or microfibers). For example, the hydrogel material such as HA may be covalently bonded to the recombinant or type I bovine collagen nanofiber of fragments thereof. In certain embodiments, the crosslinking agent generates interfacial bonding between the collagen nanofiber and the HA. Due to bonding and interaction (e.g., covalent, non-covalent or ionic bonding), the collagen nanofiber may be retained inside or inner space of the fiber-hydrogel composite (e.g., inside of the composite network). In certain embodiments, the crosslinking agent may react with the hydroxyl groups of hydrogel material (e.g. HA) and amino groups of the collagen nanofiber to form the composite network. For example, interfacial bonding between the collagen nanofiber and the hydrogel material (e.g. HA) may increase the composite stiffness even at relatively low fiber loading density.

Due to the interfacial bonding and the formation of the composite network, the fiber-hydrogel composite may have increased cell permeability and/or maintains storage modulus.

In certain embodiments, a storage modulus of the fiber-hydrogel composite is at least about 10 Pa, at least about 20 Pa, at least about 30 Pa, at least about 40 Pa, at least about 50 Pa, at least about 60 Pa, at least about 70 Pa, at least about 80 Pa, at least about 90 Pa, at least about 100 Pa, at least about 150 Pa, at least about 200 Pa, at least about 250 Pa, at least about 300 Pa, at least about 400 Pa, or at least about 500 Pa. In certain embodiments, a storage modulus of the fiber-hydrogel composite ranges from about 1 to about 1,000 Pa, from about 20 to about 800 Pa, from about 100 to about 500 Pa, or from about 150 to about 500 Pa. In certain embodiments, a storage modulus of the fiber-hydrogel composite ranges from about 0.5 to about 30 kPa. Storage modulus values as referred to herein may be determined by procedures set forth in Example 3 which follows, which include: use a analytical tool such as AR2 (TA Instruments) using a parallel plate geometry of 8 mm with 0.5 mm gap at 25° C., with linear viscoelastic region being measured for the composition sample by a strain sweep with an increasing shear strain amplitude at a set frequency (1 Hz).

In certain embodiments, as discussed, an alternate hydrogel phase such as collagen, chitosan, alginate, PVA, gelatin, PEG or other glycol ethers, cellulose, or cellulose materials may be used in place of or in combination with hyaluronic acid (HA).

In certain embodiments, the fiber-hydrogel composite may be stable at the temperature and pressure suitable for terminal sterilization (e.g., autoclaving). For example, the fiber-hydrogel composite may be stable at a temperature of about 100° C. to 131° C. for at least 30 minutes (less needed at 131° C.). In certain embodiments, the composite may have increased thermal stability and/or results in shelf stability at ambient temperatures. Thus, with autoclave sterilization for example, the gels can be sterilized at the terminal manufacturing step to reduce the cost, risk, or regulatory burden of manufacturing without significant change to the mechanical behaviors of the composite.

The preferred form of interaction between the fibers (e.g., nanofibers or microfibers) and the hydrogel component includes a crosslinking moiety, generally present in an amount effective to introduce bonding between the fibers (e.g., nanofibers or microfibers) and the hydrogel material, e.g., to induce crosslinking between collagen nanofibers and hyaluronic acid.

For example, in case that high cohesive strength is desired, the fibers (e.g., nanofibers or microfibers) and the hydrogel component may be covalently crosslinked. For example, the HA hydrogel polymer component may be covalently crosslinked to the fibers, either intramolecularly or intermolecularly or through covalent bonds. In the former case, there are no covalent bonds linking the polymers to one another or to the nanostructures, while in the latter case, there are covalent crosslinks binding the polymers to one another or to the nanostructures. The crosslinks may be formed using any suitable means, including using heat, radiation, or a chemical curing (crosslinking) agent. The degree of crosslinking should be sufficient to eliminate or at least minimize cold flow under compression. Crosslinking also includes the use of a third molecule, a “cross-linker” utilized in the cross-linking process.

“Cross-linkers” or “Cross-linking agents” may suitably include one or more selected from difunctional epoxide-based crosslinkers, 1,4-butanediol diglycidyl ether (BDDE), divinyl sulfone (DVS), PEG molecules with terminal epoxide functional groups, and HA-reactive agents. Preferably, the crosslinking agent includes DVS or BDDE. In certain embodiments, a PEG crosslinking agent, which may contain terminal functional groups, such as epoxide or vinyl sulfone groups, may be used to introduce crosslinking between the fibers (e.g., nanofibers or microfibers) and also between the fibers (e.g., nanofibers or microfibers) and the hydrogel to extend durability of the composite network and to modulate crosslinking density. In certain embodiments, the crosslinking agent does not include any spacer within its structure.

Crosslinking may also be accomplished with radiation, typically in the presence of a photoinitiator. The radiation may be ultraviolet, alpha, beta, gamma, electron beam, and x-ray radiation, although ultraviolet radiation is preferred. Useful photosensitizers are triplet sensitizers of the “hydrogen abstraction” type, and include benzophenone and substituted benzophenone and acetophenones such as benzyl dimethyl ketal, 4-acryloxybenzophenone (ABP), 1-hydroxy-cyclohexyl phenyl ketone, 2,2-diethoxyacetophenone and 2,2-dimethoxy-2-phenylaceto-phenone, substituted alpha-ketols such as 2-methyl-2-hydroxypropiophenone, benzoin ethers such as benzoin methyl ether and benzoin isopropyl ether, substituted benzoin ethers such as anisoin methyl ether, aromatic sulfonyl chlorides such as 2-naphthalene sulfonyl chloride, photoactive oximes such as 1-phenyl-1,2-propanedione-2-(O-ethoxy-carbonyl)-oxime, thioxanthones including alkyl- and halogen-substituted thioxanthonse such as 2-isopropylthioxanthone, 2-chlorothioxanthone, 2,4 dimethyl thioxanone, 2,4 dichlorothioxanone, and 2,4-diethyl thioxanone, and acyl phosphine oxides. Radiation having a wavelength of 200 to 800 nm, preferably, 200 to 500 nm, is preferred for use herein, and low intensity ultraviolet light is sufficient to induce crosslinking in most cases. However, with photosensitizers of the hydrogen abstraction type, higher intensity UV exposure may be necessary to achieve sufficient crosslinking. Such exposure can be provided by a mercury lamp processor such as those available from PPG, Fusion, Xenon, and others. Crosslinking may also be induced by irradiating with gamma radiation or an electron beam. Appropriate irradiation parameters, i.e., the type and dose of radiation used to effect crosslinking, will be apparent to those skilled in the art.

Suitable chemical curing agents, also referred to as chemical cross-linking “promoters,” include, without limitation, polymercaptans such as 2,2-dimercapto diethylether, dipentaerythritol hexa(3-mercaptopropionate), ethylene bis(3-mercaptoacetate), pentaerythritol tetra(3-mercaptopropionate), pentaerythritol tetrathioglycolate, polyethylene glycol dimercaptoacetate, polyethylene glycol di(3-mercaptopropionate), trimethylolethane tri(3-mercaptopropionate), trimethylolethane trithioglycolate, trimethylolpropane tri(3-mercaptopropionate), trimethylolpropane trithioglycolate, dithioethane, di- or trithiopropane and 1,6-hexane dithiol. The crosslinking promoter is added to the uncrosslinked hydrophilic polymer to promote covalent crosslinking thereof, or to a blend of the uncrosslinked hydrophilic polymer and the complementary oligomer, to provide crosslinking between the two components.

In certain embodiments, a concentration of the crosslinking agent (e.g., DVS, or BDDE) ranges from about 0.01 v/v %. to about 10 v/v %, 0.05 v/v %. to about 10 v/v %, 0.05 v/v %. to about 5 v/v %, from about 0.2 v/v %. to about 10 v/v %, from about 0.5 v/v %. to about 5.0 v/v %, or from about 0.5 v/v %. to about 2.5 v/v %, based on the total volume of the fiber-hydrogel composite. In certain embodiments, a concentration of the DVS ranges from about 0.01 v/v %. to about 10 v/v %, 0.05 v/v %. to about 10 v/v %, 0.05 v/v %. to about 5 v/v %, from about 0.2 v/v %. to about 10 v/v %, from about 0.5 v/v %. to about 5.0 v/v %, or from about 0.5 v/v %. to about 2.5 v/v %, based on the total volume of the fiber-hydrogel composite.

In certain embodiments, a concentration of the HA ranges from about 0.1 w/v % to about 10 w/v %, from about 0.2 w/v % to about 10 w/v %, from about 0.2 w/v % to about 5.0 w/v %, from about 0.5 w/v % to about 10.0 w/v %, from about 0.5 w/v % to about 5.0 w/v %, from about 0.5 w/v % to about 2.0 w/v %, or from about 0.8 w/v % to about 2.0 w/v %, based on the total volume of the fiber-hydrogel composite.

In certain embodiments, a concentration of the one or more ECMs ranges from about 0.1 to about 50 w/v %, from about 0.1 to about 40 w/v %, from about 0.1 to about 30 w/v %, from about 0.1 to about 20 w/v %, from about 0.1 to about 10 w/v %, from about 1 to about 10 w/v %, from about 1 to about 5 w/v %, from about 1 to about 3 w/v %, or from about 1.5 to about 3 w/v %, based on the total volume of the fiber-hydrogel composite. In certain embodiments, a fiber loading density of the collagen nanofiber ranges from about 0.1 w/v % to about 10 w/v %, from about 1 w/v % to about 10 w/v %, from about 1 w/v % to about 5 w/v %, from about 1 w/v % to about 3 w/v % based on the total volume of the fiber-hydrogel composite.

For example, in an embodiment, a concentration of the one or more ECMs ranges from about 0.1 to about 20 w/v %, a concentration of the HA ranges from about 0.5 to about 10 w/v %, and a concentration of the crosslinking agent (e.g., DVS, or BDDE) ranges from about 0.05 to about 5.0 v/v %, based on the total volume of the fiber-hydrogel composite. Further, in an embodiment, the concentration of the one or more ECMs ranges from about 1.5 to about 3.0 w/v %, the concentration of the HA ranges from about 0.8 to about 2 w/v %, and a concentration of the crosslinking agent (e.g., DVS) ranges from about 0.5 to about 2.5 v/v %, based on the total volume of the fiber-hydrogel composite.

The fiber-hydrogel composite may be formed to have a final device pH ranging from about 5.0 to about 9.0, from about 6.0 to about 8.0 from, or about 7.0 to about 7.4, in an isotonic solution.

The fiber-hydrogel composite may be formulated in a form of a sheet or a flowable or injectable fluid. For example, the fiber-hydrogel may be formulated in a solution that can be applied and shaped (or optionally dried) to form a sheet type gel. Further, the fiber-hydrogel composite may be formed in a fluid (e.g., aqueous suspension or dispersion) that can pass through of a 27-gauge or smaller needle.

In certain embodiments, the fiber-hydrogel composite may exhibit monocyte recruitment, monocyte polarization, or both. In certain embodiments, the fiber-hydrogel may accommodate or include macrophages.

In certain embodiments, the fiber-hydrogel composite is characterized in displaying a biostimulatory effect such as tissue remodeling, host cell infiltration, cell adhesion, cell migration, angiogenic responses, adipogenic responses, and macrophage polarization to pro-healing phenotypes. In particular, the fiber-hydrogel composite can have the biostimulatory effect due to the low crosslinking density of the composite materials. The fiber-hydrogel composite may have prolonged retention of biostimulatory fibers (e.g., collagen fiber) in the hyaluronic acid (HA) network.

In certain embodiments, the fiber-hydrogel composite may enable cellular infiltration. In certain embodiments, the fiber-hydrogel may accommodate or include an infiltrated macrophage.

In certain embodiments, the fiber-hydrogel may accommodate or include a M2 phenotype infiltrated macrophage because the collagen nanofiber that conditions a population of a M2 phenotype infiltrated macrophage. The fiber-hydrogel composite may modulate host cell infiltration and/or shape retention of the composite.

In certain embodiments, the fiber-hydrogel composite may have tissue remodeling effect induced by the fiber-hydrogel composite without incorporation of cells or growth factors.

In certain embodiments, the fiber-hydrogel composite may promote or induce cell migration. In certain embodiments, the fiber-hydrogel composite may attract more than 1.5-fold, more than 2.0-fold, more than 2.5-fold, more than 3.0-fold, or more than 5-fold of host cells into the injection site in vivo compared with a hydrogel control that does not include ECM nanofibers.

In certain embodiments, the fiber-hydrogel composite may promote or induce angiogenic responses. For example, the fiber-hydrogel composite promotes neo-vasculature formation. In certain embodiments, the fiber-hydrogel composite may also promote angiogenesis and soft tissue restoration in the absence of any exogenous cytokines or cells, thereby producing a long-lasting cellular repair effect.

In certain embodiments, the collagen nanofiber composites promote adipogenesis for progenitor cells in vitro and/or accelerates adipogenesis in vivo.

In certain embodiments, the collagen nanofiber composites promote adipogenesis in surrounding tissues such as the panniculus carnosus in vivo in rodent models.

In certain embodiments, the fiber-hydrogel composite is administered to a subject by injection and maintains cell viability greater than about 50%, about 60%, about 70%, about 80%, about 90%, or about 95% after 7 days of injection.

Preferably, the fiber-hydrogel composite may be formulated such that the density, ratio of gel to fibers, and other properties are variable, while maintaining sufficient porosity and strength. A ratio of the fibers (e.g., nanofibers or microfibers) to hydrogel material can be determined by any means known in the art. For example, the ratio of ECM fibers to hydrogel material (e.g. hyaluronic acid) is from about 1:100 to about 100:1 on a component-mass basis, such as about 1:50 to about 50:1, or 1:10 to about 10:1, such as 1:5 to about 5:1, such as about 1:3 to about 3:1. The ratio of ECM fibers to hydrogel material (e.g. hyaluronic acid) is also provided as a concentration basis, e.g., a given weight of polymeric fiber per volume of hydrogel material. For example, the concentration is from about 1 to 50 mg/mL. The hydrogel material is suitably generally connected, attached or disposed within the ECM fibers as forming the composite network.

The fiber-hydrogel composite may contain a plurality of pores present on or within a surface of the composite. The presence, size, distribution, frequency and other parameters of the pores can be modulated during the creation of the composite, hydrogel, or fibers (e.g., nanofibers or microfibers). Pore size can be from below about 1 nm to up to 100 μm, including 1, 2, 3, 4 5, 10, 15, 20, 30, 40, 50, 60 70, 80, 90 or 100 μm, and the size thereof may be narrowly tailored, e.g., such that at least 40%, such as 50%, 60%, 70%, 80%, 90%, 95% or greater than 95% of the pores are in a desired size or within a desired size range.

In one aspect, preferred compositions or composites comprises a plurality of pores present on a surface of a layer of the composite or composition, wherein the pores are present at a concentration of at least about 50 pores per cm² of the surface, and wherein at least 80% of the pores have an average pore diameter on the surface that is at least about 5 microns.

The fiber-hydrogel composite may be suitable for incorporation into a tissue of a human subject, and thus they are generally “biocompatible”, meaning capable of interacting with a biological system (such as found in a human subject) without inducing a pathophysiological response therein and/or thereby. In certain embodiments, the fiber-hydrogel composite is provided in order to be durably retained in the tissue, e.g., organs, nerve tissues. Alternatively, the composite may be transiently retained in the human subject and are provided as substantially biodegradable. Preferably, the ECM fibers may further include biocompatible biodegradable polymers, e.g., biocompatible biodegradable polyester.

In certain embodiments, the fiber-hydrogel composite is formed in a microbead (spherical or non-spherical) having a mean diameter in a range of about 1 μm to about 1000 μm, about 10 μm to about 1000 μm, about 20 μm to about 1000 μm, about 30 μm to about 1000 μm, about 40 μm to about 1000 μm, about 50 μm to about 1000 μm, about 50 μm to about 500 μm, about 50 μm to about 400 μm, or about 100 μm to about 500 μm. The fiber-hydrogel composite is formed into particulate formulations (e.g., microbead or microgel), enabling use of higher concentrations of each component and enhanced stability. In certain embodiments, a system of particulation may be employed wherein the pre-formed fiber-hydrogel composite is physically modulated, such as by being pushed through one, two, three, or more than three mesh screens, creating a population of non-spherical beads that are relatively similar to one another in shape and size. This multi-screen system allows for tight control over the size of the beads, thus allowing the user to modulate the size as needed. Such microbeads (e.g., non-spherical) are disclosed in US 2020/0069846. For example, the fiber-hydrogel composite is particularized by applying mechanical shear through meshes with defined sizes in the range of 50 to 400 μm.

The fiber-hydrogel composite may further include an active agent and thereby act as an active agent delivery system when applied to a body surface (e.g., a site of tissue repair) in active agent-transmitting relation thereto. The release of active agents “loaded” into the fiber-hydrogel composite (e.g., HA hydrogel or fibers) typically involves both absorption of water and desorption of the agent via a swelling-controlled diffusion mechanism. For example, active agent-containing hydrogel compositions may be employed, by way of example, in transdermal drug delivery systems, in wound dressings, in topical pharmaceutical formulations, in implanted drug delivery systems, in oral dosage forms, and the like.

In certain embodiments, the fiber-hydrogel composite may include one or more exogenous growth factors and/or cytokines. Exemplary growth factors may include, but not limited to, epidermal growth factor (EGF), platelet-derived growth factor (PDGF), vascular endothelial growth factor (VEGF), fibroblast growth factor (FGF), transforming growth factor-alpha (TGFα) and/or nerve growth factor (NGF). In certain embodiments, the fiber-hydrogel composite including EGF/TGF may advantageously be used in the acceleration of wound healing and burns, reduction in keloid scar formation (especially for burns), skin engraftment dressings, and the treatment of chronic leg ulcers. In certain embodiments, the fiber-hydrogel composite including VEGF may promote angiogenesis (blood vessel growth) or contribute to angiogenesis both indirectly and directly by stimulating proliferation of endothelial cells at the microvessel level, causing them to migrate and to alter their generic expression. In certain embodiments, fiber-hydrogel composite including FGF may promote or induce angiogenic in vivo and its angiogenicity is enhanced by combined use of TNF. For example, FGF-2 may be suitably contained in the fiber-hydrogel composite to control human megakaryocytopoiesis or stimulate endothelial cell formation and connective tissue repair. Also, keratinocyte growth factor (KGF), also known as FGF-7, may be suitably contained in the fiber-hydrogel composite for wound healing and other disorders involving epithelial cell destruction. In certain embodiments, the fiber-hydrogel composite including transforming growth factors (TGF's) may transform various cell lines, for example, may have the ability to grow in culture for more than a limited number of generations, growth in multiple layers rather than monolayers, and the acquisition of an abnormal karyotype. For example, TGF-beta may be suitably contained in the fiber-hydrogel composite to promote angiogenic effects and collagen formation in fibroblasts and antagonize the mitogenic effects of other peptide growth factors as well as inhibit the growth of many tumour cell lines.

Additional suitable active agents that may be incorporated into the fiber-hydrogel composite and delivered systemically (e.g., with a transdermal, oral, or other dosage form suitable for systemic administration of a drug) include, but are not limited to: analeptic agents; analgesic agents; anesthetic agents; antiarthritic agents; respiratory drugs, including antiasthmatic agents; anticancer agents, including antineoplastic drugs; anticholinergics; anticonvulsants; antidepressants; antidiabetic agents; antidiarrheals; antihelminthics; antihistamines; antihyperlipidemic agents; antihypertensive agents; anti-infective agents such as antibiotics and antiviral agents; antiinflammatory agents; antimigraine preparations; antinauseants; antiparkinsonism drugs; antipruritics; antipsychotics; antipyretics; antispasmodics; antitubercular agents; antiulcer agents; antiviral agents; anxiolytics; appetite suppressants; attention deficit disorder (ADD) and attention deficit hyperactivity disorder (ADHD) drugs; cardiovascular preparations including calcium channel blockers, antianginal agents, central nervous system (CNS) agents, beta-blockers and antiarrhythmic agents; central nervous system stimulants; cough and cold preparations, including decongestants; diuretics; genetic materials; herbal remedies; hormonolytics; hypnotics; hypoglycemic agents; immunosuppressive agents; leukotriene inhibitors; mitotic inhibitors; muscle relaxants; narcotic antagonists; nicotine; nutritional agents, such as vitamins, essential amino acids and fatty acids; ophthalmic drugs such as antiglaucoma agents; parasympatholytics; peptide drugs; psychostimulants; sedatives; steroids, including progestogens, estrogens, corticosteroids, androgens and anabolic agents; smoking cessation agents; sympathomimetics; tranquilizers; and vasodilators including general coronary, peripheral and cerebral. Specific active agents with which the present adhesive compositions are useful include, without limitation, anabasine, capsaicin, isosorbide dinitrate, aminostigmine, nitroglycerine, verapamil, propranolol, silabolin, foridone, clonidine, cytisine, phenazepam, nifedipine, fluacizin, and salbutamol.

In certain embodiments, the fiber-hydrogel composite may also include additional optional additive components. Such components are known in the art and can include, for example, fillers, preservatives, pH regulators, softeners, thickeners, pigments, dyes, refractive particles, stabilizers, toughening agents, detackifiers, pharmaceutical agents (e.g., antibiotics, angiogenesis promoters, antifungal agents, immunosuppressing agents, antibodies, and the like), and permeation enhancers. These additives, and amounts thereof, are selected in such a way that they do not significantly interfere with the desired chemical and physical properties of the hydrogel composition.

In certain embodiments, the fiber-hydrogel composite may include pH regulating compounds. Compounds useful as pH regulators include, but are not limited to, glycerol buffers, citrate buffers, borate buffers, phosphate buffers, or citric acid-phosphate buffers may also be included so as to ensure that the pH of the hydrogel composition is compatible with that of an individual's body surface.

In certain embodiments, the fiber-hydrogel composite may include and deliver an antibody. The term “antibody” is used herein in its broadest sense and includes certain types of immunoglobulin molecules comprising one or more antigen-binding domains that specifically bind to an antigen or epitope. For example, the fiber-hydrogel composite may include intact antibodies (e.g., intact immunoglobulins), antibody fragments, multi-specific antibodies, a monoclonal antibody, a chimeric antibody, or a humanized antibody.

In certain embodiments, the fiber-hydrogel composite may include cells for delivery. For example, the cells may be derived from the subject to whom they are administered, from a source other than the subject to whom they are administered, from a cell line, from a human source, or from a humanized animal source. In certain embodiments, the cells may include, but be not limited to, stem cells, adipose cells or tissues, or nerve cells.

In certain embodiments, the fiber-hydrogel composite may also include small molecules for delivery, which can cause pharmacological activity or anther direct effect in the diagnosis, cure, mitigation, treatment or prevention of disease or can affect the structure or function of the body.

Particularly, the fiber-hydrogel composite may further include a component that promotes angiogenesis, e.g., growth factor or cells. For example, heparin-containing hydrogel components, which can serve as growth factor binding sites to enrich and retain growth factors promoting angiogenesis and tissue formation, may also be used.

In certain embodiments, the fiber-hydrogel composite may further include one or more enzymes that may be used in the debridement of both acute and chronic wounds. Enzymes, for example, may be incorporated by directly digesting the components of slough (e.g., fibrin, bacteria, leukocytes, cell debris, serous exudate, DNA) or by dissolving the collagen “anchors” that secure the avascular tissue to the underlying wound bed.

In certain embodiments, the fiber-hydrogel composite may be delivered by any suitable method, such as via a syringe or bellows pack (single dose delivery systems) or a multidose system, such as a pressurized delivery system or delivery via a “bag in the can” type system. The administration may extend to a single dose delivery system including the fiber-hydrogel composite, for the treatment of wounds, to a pressurized delivery system including the fiber-hydrogel composite (e.g., an aerosol spray).

In certain embodiment, it may be advantageous to render the fiber-hydrogel composite electrically conductive for use in biomedical electrodes and other electrotherapy contexts, i.e., to attach an electrode or other electrically conductive member to the body surface. For example, the fiber-hydrogel composite may be used to attach a transcutaneous nerve stimulation electrode, an electrosurgical return electrode, or an EKG electrode to a patient's skin or mucosal tissue. These applications involve modification of the fiber-hydrogel composite so as to contain a conductive species. Suitable conductive species are ionically conductive electrolytes, particularly those that are normally used in the manufacture of conductive adhesives used for application to the skin or other body surface, and include ionizable inorganic salts, organic compounds, or combinations of both. Examples of ionically conductive electrolytes include, but are not limited to, ammonium sulfate, ammonium acetate, monoethanolamine acetate, diethanolamine acetate, sodium lactate, sodium citrate, magnesium acetate, magnesium sulfate, sodium acetate, calcium chloride, magnesium chloride, calcium sulfate, lithium chloride, lithium perchlorate, sodium citrate and potassium chloride, and redox couples such as a mixture of ferric and ferrous salts such as sulfates and gluconates. Preferred salts are potassium chloride, sodium chloride, magnesium sulfate, and magnesium acetate, and potassium chloride is most preferred for EKG applications. Although virtually any amount of electrolyte may be present in the adhesive compositions of the invention, it is preferable that any electrolyte present be at a concentration in the range of about 0.1 to about 15 wt. % of the hydrogel composition. The procedure described in U.S. Pat. No. 5,846,558 to Nielsen et al. for fabricating biomedical electrodes may be adapted for use with the hydrogel compositions of the invention, and the disclosure of that patent is incorporated by reference with respect to manufacturing details. Other suitable fabrication procedures may be used as well, as will be appreciated by those skilled in the art.

In some embodiments, hyaluronic acid is replaced with carboxymethyl cellulose (CMC) and collagen fibers are replaced with cellulose-based fibers. The hydroxyls on both components allow for simple modification and both can be autoclaved.

### Soft Tissue Device

Provided herein is a soft tissue device or implant that includes the fiber-hydrogel composite as described herein.

In certain embodiments, the implant may be used for promoting angiogenesis.

In certain embodiments, the implant may be used for adipose tissue formation. For example, the implant may further include biologically active material that is suitable for fat grafting, e.g., which may be differentiated into soft tissues such as fat, when supported with a suitable matrix microenvironment. In certain embodiments, the biologically active material includes a population of adipose cells, autologous adipose cells, allogenic cells, genetically modified allogenic cells, stem cells, mesenchymal stem cells, genetically modified stem cells, genetically modified allogenic induced pluripotent stem (iPS) cells, genetically modified hypoimmunogenic pluripotent stem cells, adipose stromal vascular fraction, adipose tissue, autologous adipose tissue, lipoaspirate, a derivative thereof, or a combination thereof. In some embodiments, the biologically active material includes adipose tissue.

In certain embodiments, the implant may be used for vasculature formation.

In certain embodiments, the soft tissue device or implant may suitably include the microbeads (e.g., spherical or non-spherical) made of the fiber-hydrogel composite in a volume of about 25% to 75% of the total volume of the soft tissue device. In certain embodiments, the soft tissue device or implant may suitably include the microbeads (e.g., spherical or non-spherical) in a volume of about 30% to 70% of the total volume of the soft tissue device. In certain embodiments, the soft tissue device or implant may suitably include the microbeads (e.g., spherical or non-spherical) in a volume of about 35% to 65% of the total volume of the soft tissue device. In certain embodiments, the soft tissue device or implant may suitably include the microbeads (e.g., spherical or non-spherical) in a volume of about 40% to 60% of the total volume of the soft tissue device. In certain embodiments, the soft tissue device or implant may suitably include the microbeads (e.g., spherical or non-spherical) in a volume of about 50% of the total volume of the soft tissue device.

In certain embodiments, the soft tissue device or implant may suitably include the microbeads (e.g., spherical or non-spherical) in an amount of about 10% to 90% of the total weight of the soft tissue device. In certain embodiments, the soft tissue device or implant may suitably include the microbeads (e.g., spherical or non-spherical) in an amount of about 20% to 80% of the total weight of the soft tissue device. In certain embodiments, the soft tissue device or implant may suitably include the microbeads (e.g., spherical or non-spherical) in an amount of about 30% to 70% of the total weight of the soft tissue device. In certain embodiments, the soft tissue device or implant may suitably include the microbeads (e.g., spherical or non-spherical) in an amount of about 40% to 60% of the total weight of the soft tissue device.

In certain embodiments, the microbeads are gelated or cured before the biological active material is added to the microbeads. In certain embodiments, the microbeads are gelated or cured after the biological active material is added to the microbeads. The microbeads may be gelated by chemical reaction (e.g., cross-linking) or by UV irradiation to cure or polymerize the polymers to form nanofibers.

In certain embodiments, the soft tissue device or implant may further include a compound selected from the group consisting of growth factors, compounds stimulating angiogenesis, immunomodulators, inhibitors of inflammation, and combinations thereof, which may be carried in or separately from the fiber-hydrogel composite. In certain embodiments, the soft tissue device may further include one or more compounds that have therapeutic effects, vascularization effects, anti-vascularization effects, anti-inflammatory effects, anti-bacterial effects, antihistamine effects, and combinations thereof, which may be carried in or separately from the fiber-hydrogel composite.

In certain embodiment, the soft tissue device has a tan delta value of less than about 0.27. The tan delta is the rheological loss modulus divided by the storage modulus, which means that a lower tan delta number equates to a more “solid-like” as opposed to “liquid-like” material. The tan delta may also indicate a rheological property, which may vary based on the oil or fat contents of the material or substance.

In certain embodiments, the microbeads are substantially stable at room temperature for at least about 1 week, at least about 2 weeks, at least about 1 month, at least about 2 months, at least about 3 months, at least about 4 months, at least about 5 months, or at least about 6 months.

In certain embodiments, the soft tissue device is stable at a temperature of 37° C.

The soft tissue device or implant may be used for implanting or injecting administration around the target tissues. In the soft tissue device or implant, the fiber-hydrogel composite or the microbeads (e.g., spherical or non-spherical) made of the fiber-hydrogel composite may be administered topically or subdermally.

### Kit

Provided herein is a kit including the fiber-hydrogel composite as described herein and a suitable applicator. In certain embodiments, the applicator may include an injector or injection needle.

In certain embodiments, the kit may include a vial including one or more components selected from a salt, a buffer solution, and therapeutic agent. For example, the components in the vial may include, but not limited to, lidocaine HCl in a concentration of about 3.0 mg/mL, potassium chloride in a concentration of 0.185 mg/mL, potassium phosphate monobasic in a concentration of 0.185 mg/mL, sodium chloride in a concentration of 7.40 mg/mL, and sodium phosphate dibasic in a concentration of 1.06 mg/mL.

In certain embodiments, the kit includes the fiber-hydrogel composite in a form of microbeads or microgel. Preferably, the microbeads may have a mean size a size range of 50 to 400 μm.

### Methods of Use

In an aspect, provided also are methods of producing the fiber-hydrogel composite as described herein. The method may include a step of contacting a crosslinking agent with fibers (e.g., nanofibers or microfibers) comprising one or more extracellular matrix proteins (ECMs) and hydrogel material e.g. hyaluronic acid (HA) to obtain a fiber-hydrogel composite. The hydrogel material (e.g. HA) is suitably bonded to the fibers (e.g., nanofibers or microfibers) by the crosslinking agent to form a composite network.

In certain embodiments, the method may include steps of modulating crosslinking condition. In certain embodiments, the crosslinking condition is modulated to be under a basic condition and a pH of the crosslinking condition ranges from about 9 to about 14, from about 10 to about 14, from about 10 to about 13, or from about 10 to about 12. In certain embodiments, the pH of the crosslinking condition ranges from about 10 to about 14, from about 12 to about 13.3. For example, the pH of the crosslinking condition is about 12.4, about 12.7, about 13.0 or about 13.3.

In certain embodiments, the step of contacting is performed for about 30 minutes to about 4 hours. In certain embodiments, the step of contacting is performed for more than about 30 minutes, more than about 1 hour, more than about 1.5 hour, more than about 2 hours, more than about 2.5 hours, more than about 3 hours, more than about 3.5 hours, or about 4 hours or less. In certain embodiments, the step of contacting is performed for about 30 minutes, about 1 hour, about 1.5 hour, about 2 hours, about 2.5 hours, about 3 hours, about 3.5 hours, or about 4 hours or less. In certain embodiments, the step of contacting is performed at a temperature of about 37 degrees C.

In certain embodiments, the contacting is performed for about less than about 2 hours. In such case, the contacting may be performed at elevated temperature, e.g., at a temperature of about 37 to 100 degrees C., about 40 to 100 degrees C., about 50 to 100 degrees C., or about 60 to 100 degrees C.

In certain embodiments, the contacting is performed for more than one day. In such case, the contacting may be performed at lower temperature, e.g., at a temperature of about 0 to 37 degrees C., about 0 to 30 degrees C., about 0 to 25 degrees C., about 0 to 20 degrees C., or about 0 to 15 degrees C.

The method of producing the fiber-hydrogel composite may further include a step of swelling the fiber-hydrogel composite. In certain embodiments, the step of swelling is performed by incubating the fiber-hydrogel composite in an aqueous buffer solution (e.g., PBS).

In certain embodiments, the buffer solution may be used in greater than 10 times, greater than 50 times, greater than 100 times, greater than 200 times, greater than 300 times, greater than 400 times, greater than 500 times, or greater than 1000 times, of volume of the fiber-hydrogel composite. In certain embodiments, the swelling is performed (continuously or discontinuously) for more than an hour, more than 2 hours, more than 5 hours, more than 10 hours, more than 12 hours, more than 15 hours, more than 20 hours, more than 24 hours, more than 30 hours, more than 40 hours, more than 50 hours, more than 60 hours, or more than 72 hours. Preferably, the swelling is performed (continuously or discontinuously) for 24 to 72 hours at room temperature.

The method of producing the fiber-hydrogel composite may further include processing the fiber-hydrogel composite to particularize into to microbeads. In certain embodiments, the microbeads have a mean diameter in a range of about 1 μm to about 1000 μm, about 10 μm to about 1000 μm, about 20 μm to about 1000 μm, about 30 μm to about 1000 μm, about 40 μm to about 1000 μm, about 50 μm to about 1000 μm, about 50 μm to about 500 μm, about 50 μm to about 400 μm, or about 100 μm to about 500 μm. As described above, the fiber-hydrogel composite is formed into particulate formulations (e.g., microbead or microgel), enabling use of higher concentrations of each component and enhanced stability. In certain embodiments, a system of particulation may be employed wherein the pre-formed fiber-hydrogel composite is physically modulated, such as by being pushed through one, two, three, or more than three mesh screens, creating a population of nonspherical beads that are relatively similar to one another in shape and size. In certain embodiments, the processing preferably may include mechanical milling or mechanical screening by applying shear using a mesh. This two-screen system allows for tight control over the size of the beads, thus allowing the user to modulate the size as needed. Such microbeads (e.g., non-spherical) are disclosed in US 2020/0069846. For example, the fiber-hydrogel composite is particularized by applying mechanical shear through meshes with defined sizes in the range of 50 to 400 μm.

In certain embodiments, the fiber-hydrogel composite produced herein may be in a biphasic gel or monophasic gel. For example, the fiber-hydrogel composite is a biphasic gel with similar bead sizes made by forcing the gel through screens. Alternatively, the fiber-hydrogel composite is a monophasic gel with a continuous distribution of gel bead sizes made by homogenizing the gel or mechanically disrupting the gel during the crosslinking reaction.

The method of producing the fiber-hydrogel composite may further include sterilizing the fiber-hydrogel composite. Any sterilizing method or process in the art may be used without limitation. For example, sterilizing the packaged composite to autoclave at 118° C. for about 5 to 30 minutes.

The method of producing the fiber-hydrogel composite may further include fabricating the fiber-hydrogel composite in a sheet or an injectable fluid.

In certain embodiments, the method may further comprise incubating an infiltrated macrophage in the fiber-hydrogel composite. In certain embodiments, the fiber-hydrogel composite including the collagen nanofiber may condition a population of a M2 phenotype infiltrated macrophage.

Provided also are methods of using the fiber-hydrogel composite to treat a subject.

In one aspect, the disclosure provides a method of forming adipose tissue formation in a subject. The method includes administering the fiber-hydrogel composite as described herein to the subject. In the subject of prolonged in vivo retention of fiber-hydrogel composite or enhanced host cell infiltration is induced by the fiber-hydrogel composite. In certain embodiments, the fiber-hydrogel composite includes cells or growth factors. In certain embodiments, the fiber-hydrogel composite does not include cells or growth factors.

In certain embodiments, the subject exhibits neo-vasculature formation facilitated by M2 macrophage polarization. In certain embodiments, the subject exhibits an increase in α-SMA+ cells around 2.5-fold at the site of administration. In certain embodiments, the subject exhibits localization of endothelial cells and CD163+ M2 macrophages at the site of administration. In certain embodiments, the subject displays an increase of around 1.5-fold of CD68+ pan-macrophages at the site of administration by post-operative day 7. In certain embodiments, the subject displays an increase of around 2-fold of CD68+ pan-macrophages at the site of administration by post-operative day 14.

In an aspect, the disclosure also provides a method of delivering a cell or tissue in a subject. The method includes encapsulating one or more cells or tissues in the fiber-hydrogel composite as described herein to form a suspension; and applying the suspension to a target site in the subject.

In another aspect, the disclosure also provides a method of delivering adipose tissue in a subject. The method includes encapsulating one or more adipose tissues in the fiber-hydrogel composite as described herein to form a suspension; and applying the suspension to a target site in the subject. In certain embodiments, the one or more adipose tissues suitably include adipose-derived stem cells, adipocytes, or combinations thereof.

In an aspect, the disclosure also provides a method of delivering a pharmaceutical agent in a subject. The method includes combining a pharmaceutical agent and the fiber-hydrogel composite as described herein to form a mixture; and applying the mixture to an intended site of delivery.

As discussed above, the fiber-hydrogel composite as described herein can be used advantageously in numerous tissue repair situations, as well as in other applications, such as providing coatings on catheters and other surgical devices and implants. The fiber-hydrogel composite can also be used to deliver active agents described herein, such as antibiotics, growth factors, and immunosuppressive agents. For example, the disclosure provides a method for healing a soft tissue defect comprising applying the fiber-hydrogel composite to a soft tissue defect.

It will be appreciated that advantageous properties of the fiber-hydrogel composite described herein include the ability to: 1) provide easy characterization and quality control; 2) integrate with existing tissue matrices; 3) directly incorporate into newly formed matrices; 4) directly include cells and bioactive factors; 5) maintain biocompatibility; 6) control bioresorption; 7) cast easily into complicated anatomical shapes due to greater structural rigidity owing to the nanostructures; 8) exhibit the mechanical properties of native tissues such as articular cartilage; 9) crosslink fibers and hydrogel phase in one step; and 10) retain mechanical properties after terminal sterilization by autoclave.

In another aspect, the fiber-hydrogel composite can be used to repair cartilage tissue. Current biologically-based surgical procedures for cartilage repair include autologous chondrocyte implantation, drilling, abrasion chondroplasty, microfracture, and mosaic arthroplasty. All these procedures treat only focal articular cartilage injuries, and not cartilage denuded joint surfaces such as seen in severe osteoarthritis and rheumatoid arthritis. Also, they use either cartilage tissue plugs or expanded chondrocytes harvested from the patient to fill cartilage defects. These tissues or chondrocytes are expected to fill the defect by synthesizing entirely de novo material, such as newly synthesized hyaline cartilage, that has integrated with existing cartilage matrices and has the biomechanical properties of normal cartilage. However, such procedures all promote the formation of a reparative tissue (fibrocartilage) rather than true hyaline cartilage with further mechanical damage to fibrocartilage thought to predispose the joint to osteoarthritis. Furthermore, the availability of endogenous cartilage as a repair material is quite limited with its acquisition presenting its own risks and morbidity to the patient. As evident from the foregoing discussion, the resulting hydrogel/nanofiber compositions disclosed herein present practical materials for promising new therapies in patients suffering from cartilage degenerative diseases.

As described herein, the fiber-hydrogel composite can be prepared having widely varying properties that are suitable for any number of synthetic tissue implantation or augmentation, as well as other clinical applications. As already described, the fiber-hydrogel composite can be used to repair cartilage defects produced as a result of either injury or disease. Defects due to injury that can be so repaired can be sports- or accident-related, and may involve only the superficial cartilage layer, or may include the underlying subchondral bone. Defects due to disease which can be repaired using the compositions described herein include those resulting from osteoarthritis and rheumatoid arthritis. Whether from injury or disease, such defects may be in either mature or growth plate cartilage. Formulations for hydrogels for synthetic growth plate cartilage may require the inclusion of unsubstituted scaffold material to allow for controlled bioresorption of the biomaterial during growth.

Another field where the fiber-hydrogel composite described herein can be useful is the repair, reconstruction or augmentation of cartilaginous as well as soft tissues of the head and neck. The availability of biomaterials for soft tissue augmentation and head and neck reconstruction has remained a fundamental challenge in the field of plastic and reconstructive surgery. Significant research and investment has been undertaken for the development of a material with appropriate biological compatibility and life span. The outcomes of this research have not been promising. When placed in immunocompetent animals the structural integrity of the fiber-hydrogel composite has been shown to fail as the framework is absorbed. Furthermore, though conventional synthetic materials offer excellent lifespan, they presented certain unavoidable pitfalls. For example, silicones have been fraught with concerns of safety and long-term immune related effects. Synthetic polymers PTFE (gore-tex) and silastic offer less tissue reactivity but do not offer tissue integration and can represent long term risks of foreign body infections and extrusion. The fiber-hydrogel composite will be useful to prepare a synthetic soft-tissue scaffold material for the augmentation or repair of soft-tissue defects of the head and neck. In particular, the hydrogel/nanofiber compositions, which are non-inflammatory, non-immunogenic, and which can be prepared having the appropriate degree of viscoelasticity (see description herein), could be used as an effective implantable scaffold material. In addition, the fiber-hydrogel composite can be used, for example, as a novel, biocompatible and biocompliant materials to prepare cartilage implants which are frequently used in reconstructive procedures of the head and neck to repair cartilaginous or bony defects secondary to trauma or congenital abnormalities. Applications specific to the ear include otoplasty and auricular reconstruction, which are often undertaken to repair cartilaginous defects due to trauma, neoplasm (i.e., squamous cell carcinoma, basal cell carcinoma, and melanoma), and congenital defects such as microtia. Applications specific to the nose include cosmetic and reconstructive procedures of the nose and nasal septum. Dorsal hump augmentation, tip, shield and spreader grafts are frequently used in cosmetic rhinoplasty. Nasal reconstruction following trauma, neoplasm, autoimmune diseases such as Wegeners granulomatosis, or congenital defects require cartilage for repair. Septal perforations are difficult to manage and often fail treatment. Cartilage grafts would be ideal for these applications, as autologous or donor cartilage is often unavailable. Applications specific to the throat include laryngotracheal reconstruction, which in children usually requires harvesting costal cartilage, which is not without morbidity. Auricular and septal cartilage is often inadequate for this application. Synthetic cartilaginous materials prepared from hydrogels disclosed herein can be synthesized to suit each of the foregoing applications, based on tuning parameters of hydrogel synthesis such as reagent concentration, substitution and cross-linking rates. Laryngotracheal reconstruction is usually performed for airway narrowing due to subglottic or tracheal stenosis. The etiology may be traumatic (i.e., intubation trauma, or tracheotomy) or idiopathic. Other possibilities include chin and cheek augmentation, and use in ectropion repair of the lower eyelid, in addition to numerous craniofacial applications. It should be noted that these applications may not need cartilage with the exacting mechanical properties of articular cartilage. Inclusion of a cell population or bioactive agents may also be desirable.

The fiber-hydrogel composite described herein can be used for repair and narrowing of the nasal cavity, normally following overly aggressive surgical resection, to prevent the chronic pooling of fluid in the nasal passages that leads to infection and encrustation. Another promising application is in laryngotracheal reconstruction in both children and adults, as a result of laryngotracheal injury due for example to intubation during a surgical procedure such as cardiovascular surgery. The fiber-hydrogel composite as herein described also can be used to provide cricoid ring replacements to protect the carotid artery following neck resection for cancer—the fiber-hydrogel composite can be placed between the carotid artery and the skin as a protective barrier for the carotid artery against loss of the skin barrier. As a protective coating during neuronal repopulation of a resected nerve—often fibrous tissue forms faster than the neuronal repopulation preventing its eventual formation. Placement of the nerve ends within the fiber-hydrogel composite pre-cast tube could exclude fibrous tissue formation from the site of repopulation.

The fiber-hydrogel composite can also be used for repair of soft tissue defects of any internal or external organs. For example, the fiber-hydrogel composite can be used to for chin and cheek augmentation, and use in ectropion repair of the lower eyelid, in addition to numerous craniofacial applications. For cosmetic and reconstructive purposes in sites other than the head and neck, for example use as breast implants for breast augmentation, as a wound sealant, for example to fill the void left after removal of lymph nodes (i.e. due to cancer) in the breast or neck, to seal the lymphatics and abate uncontrolled fluid drainage into the resection site that may lead to infection and other complications.

In addition to the above uses, the fiber-hydrogel composite described herein can be used in other tissue engineering applications to produce synthetic orthopaedic tissues, including, but not limited to, bone, tendon, ligament, meniscus and intervertebral disc, using similar strategies and methodologies as described above for the synthesis of artificial forms of cartilage. The fiber-hydrogel composite also can be used to make synthetic non-orthopaedic tissues including but not limited to vocal cord, vitreous, heart valves, liver, pancreas and kidney, using similar strategies and methodologies as described above for the synthesis of artificial forms of cartilage.

Another field where the fiber-hydrogel composite disclosed herein can be used is in gastrointestinal applications where it is necessary to treat or prevent the formation of scar tissue or strictures in abdominal or gastrointestinal organs. There already are a number of products at various stages of clinical and FDA approval, which generally are termed “hydrogels,” that are designed or intended to be useful in the treatment and prevention of scarring and/or stricture formation. The fiber-hydrogel composite is superior to other known hydrogels in that the ones disclosed here can include a nanostructure which can provide support, shape, and strength to hydrogel materials. The fiber-hydrogel composite disclosed herein can be used in similar applications as the already known hydrogels are used or intended to be used, including the following: for treatment of strictures or scarring of the gastrointestinal tract. The treatment involves injection of the fiber-hydrogel composite at the site of an anticipated stricture to prevent scarring, or at a site of existing stricture after therapy to enlarge the narrowed GI tract to prevent the stricture from reoccurring.

The fiber-hydrogel composite as described herein can also be used for the treatment of esophageal strictures. Esophageal strictures are a common complication of gastroesophageal reflux disease (GERD). GERD is caused by acid, bile and other injurious gastric contents refluxing into the esophagus and injuring the esophageal lining cells. Approximately 7-23% of GERD patients develop an esophageal stricture, or fibrous scarring of the esophagus. Esophageal scarring also can be caused by ablative therapies used to treat Barrett's esophagus. The major complication of such ablative therapies is that the ablative injury extends too deeply into the esophageal wall and results in an esophageal scar or stricture. Esophageal strictures prevent normal swallowing and are a major cause of patient morbidity. The materials described herein may be used to treat or prevent esophageal strictures resulting from GERD, Barrett's esophagus, and esophageal ablative therapies.

The fiber-hydrogel composite may also be used for treatment of Crohn's disease. Crohn's disease causes strictures or scars that block off or narrow the lumen of the bowel, preventing normal bowel function. For example, the fiber-hydrogel composite may be useful to treat or prevent such strictures.

The fiber-hydrogel composite can also be used in methods for treating primary sclerosing cholangitis (PSC). PSC is a rare disease of the bile ducts of the liver. The bile ducts form a branching network within the liver and exit the liver via two main branches that are combined into the common bile duct which drains the liver and gallbladder of bile into the duodenum. The bile ducts are very narrow in diameter, measuring only up to 2 mm normally at their largest most distal portions, and yet they must normally drain liters of bile every day from the liver into the duodenum. Any blockage of these ducts can result in a serious condition known as jaundice, which allows many toxins and especially hemoglobin breakdown products to accumulate in the body. PSC is a scarring or structuring disease of the bile ducts within the liver and in the extrahepatic bile ducts described above that connect the liver to the small intestine. The bile duct strictures of PSC may be treated or prevented with the present hydrogel/nanofiber compositions.

The fiber-hydrogel composite can also be used to treat chronic pancreatitis. Chronic pancreatitis is a chronic inflammatory disease of the pancreas that may be complicated by scars or strictures of the pancreatic ducts. These strictures block the drainage of pancreatic juice, which normally must exit the pancreas through a system of ducts or drainage conduits into the small intestine. The pancreatic juice contains many digestive enzymes and other elements important to normal digestion and nutrient absorption. Blockage or narrowing of the pancreatic ducts by chronic pancreatitis can results in severe complications in which the pancreas autodigests and forms life-threatening abdominal infections and or abscesses. The pancreatic strictures of chronic pancreatitis may be treated or prevented with the present hydrogels.

The fiber-hydrogel composite may also be used for treatment of gallstone-induced bile duct and pancreatic duct strictures. Gallstones are a very common disorder, a principal complication of which is the formation of bile duct and pancreatic duct strictures, which may be treated or prevented with the hydrogels. for treatment of ischemic bowel disease. The intestines are prone to the formation of scars or strictures when their blood supply is compromised. Compromised blood flow is called ischemia, and can be caused by many pathologies, including cardiovascular disease, atherosclerosis, hypotension, hypovolemia, renal or hepatic disease-induced hypoalbuminemia, vasculitis, drug-induced disease, and many others. The end stage result of all of these etiologies can result in intestinal strictures that block off the bowel and prevent its normal function. The present hydrogel/nanofiber composites may be used to treat or prevent ischemic bowel strictures.

The fiber-hydrogel composite may also be used for treatment of radiation-induced intestinal strictures. Radiation therapy for cancer is associated with numerous morbidities, important among which is intestinal stricture formation. The fiber-hydrogel composite may be used to treat or prevent radiation-induced intestinal strictures.

In addition to making synthetic tissues or repairing native tissues, the fiber-hydrogel composite disclosed here also can be used to provide a coating for non-biological structures or devices to be used in surgery or otherwise for in vivo implantation, such as surgical instruments, or ceramic or metal prostheses. Such a coating would provide a barrier between the non-biologic device material and living tissue. The role of fiber-hydrogel composite as a barrier for non-biologic devices includes, but is not limited to: 1) prevention of absorption of macromolecules and/or cells on the surfaces of non-biologic devices, which can lead to protein fouling or thrombosis at the device surface; 2) presentation of a non-toxic, non-inflammatory, non-immunogenic, biologically compatible surface for devices made from otherwise non-biologically compatible materials; 3) compatibility with device function such as diffusion of glucose for a glucose sensor, transmission of mechanical force for a pressure sensor, or endothelization of a vascular graft or stent; 4) enhancement of device function, such as providing a charge barrier to an existing size barrier in a MEMS based artificial nephron; 5) incorporation into non-biologic devices of a viable cell population entrapped within an aqueous, physiologically compatible environment; and 6) inclusion of drugs or bioactive factors such as growth factors, antiviral agents, antibiotics, or adhesion molecules designed to encourage vascularization, epithelization or endothelization of the device.

Based on the foregoing, the fiber-hydrogel composite may be used to provide a non-allergenic coating for a variety of implantable devices including an implantable glucose sensor for management of diabetes. In addition, the fiber-hydrogel composite may be used to provide: a charge barrier for the development of MEMS-based artificial nephrons; an aqueous, physiologically compatible environment in which embedded kidney cells such as podocytes can be incorporated into a MEMS-based artificial nephron design; and a coating for implantable MEMS devices designed for a variety of purposes including, but not limited to, drug delivery, mechanical sensing, and as a bio-detection system.

The disclosed fiber-hydrogel composite, and particularly including HA, also may be covalently attached to silicon-based devices, e.g. through first covalent attachment of the primary amine of tyramine to the silicon surface to provide a hydroxyphenyl coated surface chemistry. This may use the same chemistry used to bind DNA that has been modified with a free amine to silicon surfaces. The HA-based fiber-hydrogel composite then is covalently coupled to the hydroxyphenyl coated surface by the same peroxidase driven chemistry used in its preferred cross-linking mode described above.

The fiber-hydrogel composite also can be used for coating non-biologic cardiovascular devices such as catheters, stents and vascular grafts. These would include devices made from materials conventionally not used because of their biological incompatibility, but which have superior design characteristics to those devices currently in use. Bioactive factors could be incorporated into the hydrogels to promote endothelization or epithelization of the hydrogel, and thus of the implanted device.

Although particular examples and uses for the hydrogel/nanostructure composites of the invention have been described herein, such specific uses are not meant to be limiting. The hydrogel/nanostructure composites of the invention can be used for any application generally used for known hydrogels, and in particular, are useful for the repair and/or regeneration of soft tissue anywhere in the body.

### EXAMPLES

### Introduction

Crosslinked hydrogels incorporating bioactive components are promising as adipose tissue substitute in soft tissue repair. Collagen, as a bio-active and bio-stimulatory component, was extensively used as fillers and wound dressing, but the drawback was its volume retention and the resultant inflammatory responses. In this disclosure, a nanofiber-hydrogel composite (NHC) incorporating bio-stimulatory collagen nanofibers and hyaluronic acid (HA) network with mechanical reinforcement was reported. By tuning the crosslinking conditions and collagen nanofiber loading density, we developed a NHC construct that can attract more than 2.5-fold host cells into the injection site in vivo compared with the hydrogel controls. Furthermore, the prolonged stimulatory effect by retaining NHC conditioned the infiltrating macrophages towards pro-regenerative M2 phenotype, facilitating the neo-vasculature formation. In consequence, an improved adipose tissue formation was observed in the NHC close to the vasculature formation as a sign for accelerated tissue remodeling, which highlights the advantages of a prolonged retention of bio-stimulatory collagen fibers in a hyaluronic acid (HA) network and provide new approach to soft tissue engineering. Surgeries like tumor excision and injury repair as well as burn damage often lead to adipose tissue loss. The absence of such tissue may cause reduced functionality and inconvenience to patients [1, 2]. Conventional means to restore adipose tissue have specific drawbacks and are limited by their cost and potential morbidity if surgeries are involved. Autologous tissue repair that requires surgery to transfer tissues from another part of the body is prone to deformity at the donor site [3]. Allogeneic grafting may be associated with a number of potentially fatal complications such as graft failure and graft to host disease [4]. Synthetic implants, even though they have been more accepted in adipose tissue repair, still have the disadvantages of foreign body response, followed by necrosis of the host tissue [5].

Naturally derived materials such as collagen, silk and extracellular matrix have been processed as scaffolds to augment adipose tissue reconstruction [6, 7]. These materials are highlighted by their biocompatibility and controllable degradation rates. Various studies have shown scaffolds developed from these materials have robust bio-stimulatory effect to attract cellular ingrowth, but with numerous trade-offs, including high lot-to-lot variability, limited control of design parameters, high regulatory scrutiny, high cost, and possible compromised deformation or structural integrity [6].

Hydrogels, due to their biomimetic features close to the tissue microenvironment and the ability to be tailored for different adipose tissue needs such as volume and porosity, are the most popular scaffold matrices for tissue restoration in the field of soft tissue repair [8, 9]. Various studies have shown that hydrogel scaffolds with similar storage moduli (G′) to the native soft tissue (100- to 500-Pa) could facilitate volume and shape retention [10, 11]. However, the resilient and viscoelastic properties of hydrogels are at the expense of the high degree of crosslinking density, which reduces the rate of cell infiltration and host tissue ingrowth if the hydrogels are poorly biodegradable, leading to the compromised regeneration and tissue remodeling [12]. Among various hydrogel products, hyaluronic acid (HA) has been widely used as fillers and foams for soft tissue augmentation because of its biocompatibility as an endogenous polysaccharide, its moderate biodegradability, and similar physical properties to the soft tissues like adipose tissue, fibrous tissue, and nerves [13, 14].

Studies have shown that skewed polarization of macrophages activated by the mechanical properties of the scaffolds can predict the degree of constructive remodeling from fibrosis response to matrix remodeling (M1 macrophage-dominant pro-inflammatory response vs. M2 macrophage-dominant pro-regeneration response) [15, 16]. Therefore, the ability of the hydrogel scaffolds to promote macrophage infiltration and programming in acute and chronic inflammation is crucial during tissue remodeling. Electrospun nanofiber meshes have been widely used as regenerative substrates to mimic the extracellular matrix of the host tissue [17]. Studies have shown a M2-favored macrophage polarization when the macrophages are interacting with the electrospun fiber meshes [18]. However, such nanofiber mat devices have numerous disadvantages. As solid 2-dimensional mats, the scaffolds are not injectable, inherently anisotropic in mechanical properties, and are not suitable for volumetric applications. There are also constraints on device design options, since the variables are interrelated. For example, for mats, the scaffold porosity is dictated by the fiber diameter and orientation, which also drives the scaffold mechanical priorities, so that mechanical properties and porosity cannot be easily modified independently from one another.

Thus, to maximize the therapeutic effect, earlier studies from our group presented a robust therapeutic system formulated by hyaluronic acid (HA) chemically bonded to poly(F-caprolactone) (PCL) nanofibers, which gives the material higher porosity and cell permeability while maintaining storage moduli to match the adipose tissue [19, 20]. In this study, to fulfill the critical requirements for macrophage attraction and programming, host endothelial cell infiltration and distribution and final adipose tissue formation, we report the synthesis and characterization of a hydrogel-nanofiber composite system that harnesses hyaluronic acid (HA) covalently bonded to electrospun type I bovine collagen nanofiber fragments. Using divinyl sulfone (DVS) as the crosslinker which reacts with the hydroxyl and amino groups, we introduced interfacial bonding between the HA hydrogel and the collagen nanofibers to form a composite network [21]. This system takes advantage of the improved mechanical properties over the mixture of nanofibers and the hydrogels, the excellent injectability by the loose crosslinking density and the feasibility of terminal sterilization by autoclaving. The biocompatibility, cell adhesion and cell migration potentials of the materials were assessed in vitro using human mesenchymal stem cells (MSCs) and human umbilical vein endothelial cells (HUVECs). In vivo studies were performed using subcutaneous injections on the back of SD rats, where H/E staining was utilized to analyze host cell infiltration, immunofluorescence with various markers were harnessed to investigate the macrophage attraction and conditioning, angiogenesis, and adipose tissue regeneration.

### Example 1: Collagen Fiber Production

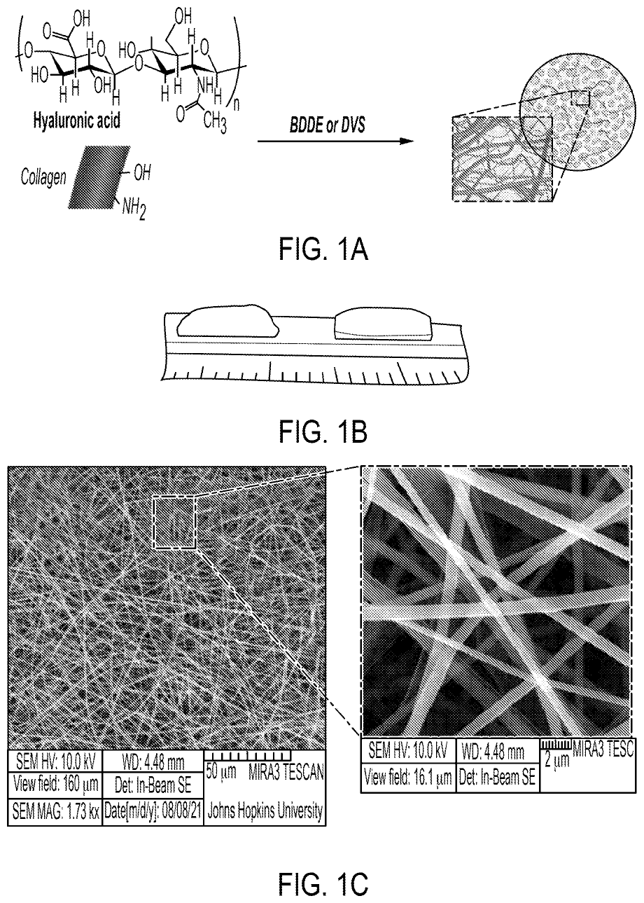

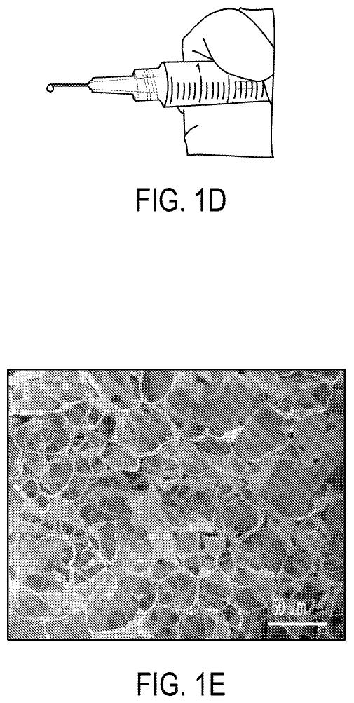

Bovine source type I collagen solution was purchased from Advanced Biomatrix. Bovine collagen solution was firstly lyophilized overnight to obtain collagen powders, and then type I collagen solution (8 w/v %) was prepared in 1,1,1,3,3,3-hexafluoro-2-propanol (HFIP) at room temperature for around 6 hours to make a viscous cloudy electrospinning solution. The electrospinning was performed with the following parameters: 5 mL/h of the flow rate; 20-25 kV of the voltage applied to the 22-G metallic needle; 12.5 cm of the collecting distance; 900 rpm of the rotation rate of the metallic collector. This set of parameters results in a mean fiber diameter of around 600 nm (FIG. 1C). By using carbodiimide chemistry, fibers were immersed in ethanol solution (95% v/v %) containing 50 mM 1-Ethyl-3-(3-dimethylaminopropyl) carbodiimide (EDC) and 20 mM N-hydroxysuccinimide (NHS) for 24 hours. After the crosslinking, fibers were washed in 0.75% glycine solution three times with 5 minutes each time to remove the excessive reagents and to quench the activated fiber surface. The collagen fibers were then broken down to fragments using cryomilling (Freezer/Mill 6770, SPEX SamplePrep). The fragments were filtered through different cell strainers (40 and 100 μm) to reach a relative uniform fiber length.

### Example 2: Preparation of the Collagen Fiber-HA Hydrogel Composite

The proposed NHC construct is composed of three components: hyaluronic acid (HA) network, bovine type I collagen nanofibers, and divinyl sulfone (DVS) crosslinker (FIG. 1A). Before incorporating the collagen nanofibers (produced in example 1) into the HA network during crosslinking, we first optimized the crosslinking conditions for the HA gel phase alone Sodium hyaluronate (MW 1.5 MDa) was purchased from LifeCore. HUVECs and vascular endothelial cell culture medium were purchased from Lonza. All other chemical reagents were purchased from Sigma-Aldrich. All other cell culture reagents and supplements were obtained from Invitrogen. HA was dissolved in distilled water at a stock concentration of 25 mg/mL. DVS concentration was calculated as the ratio to the hydroxyl groups in HA (such as 1.17 w/v %, 2.34 w/v %, and 4.68 w/v %). The stock HA solution was diluted to 2 w/v % using distilled water and sodium hydroxide to get four different pHs (12.4, 12.7, 13.0 and 13.3), with other parameters set to be the same (2 w/v % HA, 37° C., 3-hour reaction time). The reaction time or gelation kinetics were performed by preparing multiple samples and measuring the mechanical properties at various timepoints (30 min, 1 hour, 2 hours, 3 hours, 4 hours, 8 hours and 16 hours) to get a timepoint where the stiffness reaches a plateau. The crosslinking of the NHCs followed the same conditions to the HA hydrogels, different fiber density (0, 1 and 3 w/v %) were added to the mixed precursors to test the gelation kinetics of the NHCs. After the gelation of hydrogels and NHCs, dialysis was performed using dialysis membranes (6000-8000 MWCO, Spectrum) against pH 7.4 phosphate buffer for 48 hours to remove the unreacted DVS, to balance the pH and to swell the samples for further studies. The mechanical properties were measured again after the swelling. Microgels were then generated with stainless steel wire cloth discs to reach a gel particle size at around 100 μm as we previously reported.

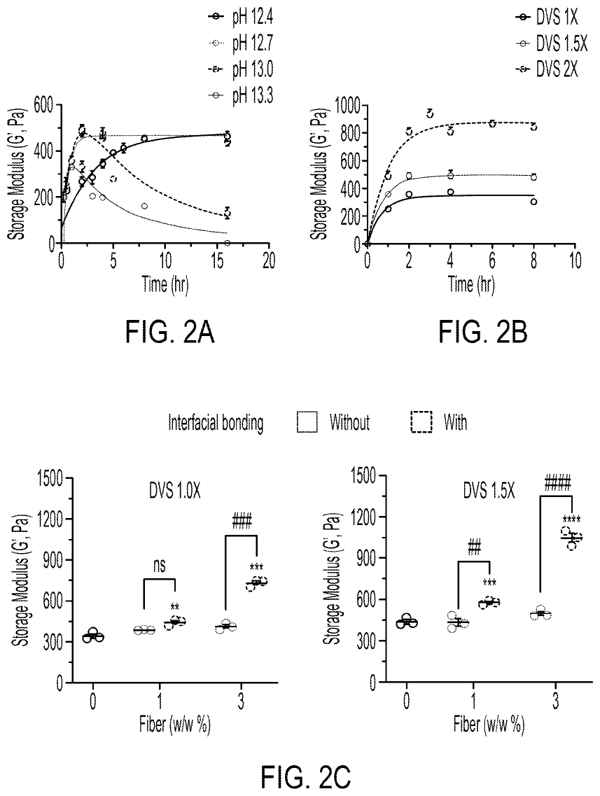

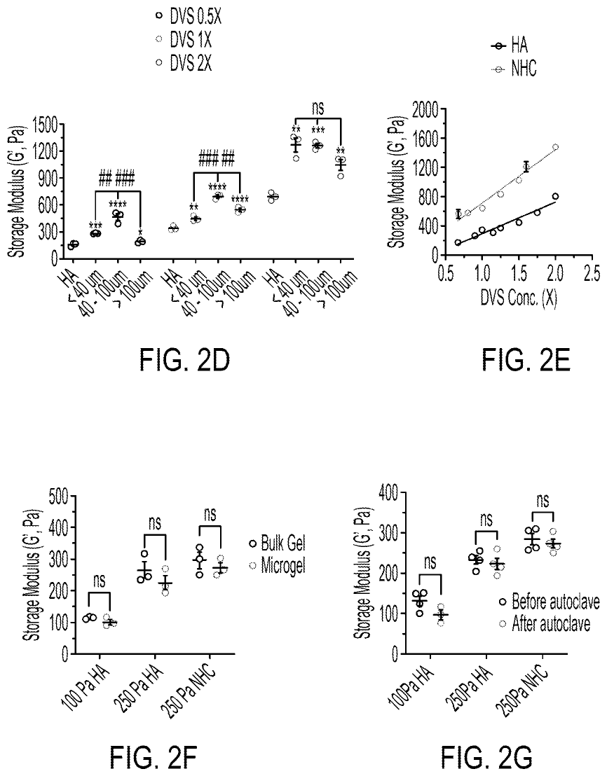

We found that using DVS chemistry to crosslink the HA network at a pH of 12.7 was effective in forming a robust crosslinked hydrogel while minimizing the degradation of HA molecules and collagen fibers during gelation (FIG. 2A). The reaction pH had a large effect upon the resulting gel in the range of pH 12-13.3. With other parameters set to be the same (37° C., HA concentration 2 w/v %, DVS concentration 2.93 w/v %), while the reaction pH was set to be 12.4, 12.7, 13.0 and 13.3 prepared by different NaOH concentrations (0.001 M, 0.01 M, 0.1 M, 1 M). The reaction at pH 13.3 and 13.0 showed a dramatic degradation after 2 hours of reaction, resulting in two unstable hydrogels with low reproducibility.

The crosslinking time was optimized to around 2 hours to reach the maximal storage modulus and limit degradation (FIG. 2B). After tuning the DVS chemistry, we introduced the nanofibers to the HA network while crosslinking to generate interfacial bonding between the HA network and the nanofibers, and a reinforcement effect was observed comparing HA and NHC at a similar crosslinking density (FIG. 1B). The close association between fibers and gel phase results in a robust, porous gel (FIG. 1E). Additionally, the composite could pass through 27-gauge needle easily after crosslinking (FIG. 1D). To further investigate this reinforcement effect quantitatively, we measured the storage modulus G0′ (normalized to a storage modulus control) of the HA hydrogel phase, the G′ of the overall NHC and the G′ of a hydrogel-nanofiber mixture without interfacial bonding. In a rheological test with 1 to 3 w/v % of fiber loading density, the G′ of the composite was ranging from 1.5 to 4 folds higher than that without interfacial bonding, and the G′ difference increased with the increase of the fiber loading and the crosslinker concentration (FIG. 2C). Furthermore, we also investigated the effect of fiber lengths on the stiffness enhancement by using different cell strainers (40 μm, 100 μm, unscreened) to screen out the large fiber fragments. In an unintuitive result, the gels with the fiber fragments with length between 40 μm to 100 μm helped generate the largest stiffness enhancement (FIG. 2D), though this relative enhancement was minimized at the highest crosslinking concentrations.

In consequence, these tuning steps allowed us to generate NHC with G′ in the range of 450 Pa to 1500 Pa after crosslinking (FIG. 2E), and 150-Pa to 1000-Pa after swelling. To mimic the soft tissue microenvironment, HA controls (G′=100-Pa and G′=250-Pa) and composite (G′=250-Pa and G0′=100-Pa) were generated and particularized to microgels with diameters around 100 μm (FIG. 1A). The storage modulus for all three groups were measured not significantly different from the initial crosslinked bulk gels (FIG. 2F). Lastly, before utilizing the prepared materials for later in vitro and in vivo studies, we autoclaved the hydrogels and composites and observed that the G′ measurements were not significantly lowered after the terminal sterilization, indicating the translational potential of this material. Autoclaving was performed to sterilize the hydrogels and NHCs after gelation. Briefly, the gels were placed at the autoclave cycle at 118° C. with a 5-minute sterilization step. The total sterilization cycle would take 30 minutes to complete [22]. After the sterilization, the mechanical properties of each gel were measured again using rheological tests, demonstrating excellent thermal stability, enabling terminal sterilization and a robust shelf life (FIG. 2G).

### Example 3: Mechanical Characterization of NHC

The rheological characterizations of hydrogels and NHCs were performed as described previously. Rheometry (AR2, TA Instruments) was harnessed to measure various shear mechanical properties of hydrogels and NHCs using a parallel plate geometry of 8 mm with 0.5 mm gap at 25° C. A linear viscoelastic region was measured for the gels by a strain sweep with an increasing shear strain amplitude at a set frequency (1 Hz). The three main properties were storage modulus (G′), loss modulus (G″) and tan delta (G″/G′) (FIG. 2A-G). The injection forces of bulk gels and microgels of HA and NHC were measured by extruding 1 mL sample through a 27-gauge NIPRO needle in 1 mL BD syringe using Instron load frame (34SC-05, Norwood, MA). Statistical analysis Data are shown as means±SEM. Data were analyzed using GraphPad Prism software (GraphPad Software Inc.) by Student's t test (unpaired and two-tailed), one- or two-way ANOVA (analysis of variance), followed by Dunnett's or Bonferroni's post hoc test as needed. The values were considered significantly different at P<0.05.

### Example 4: In Vitro Characterization of NHC

### Cell Culture

Human umbilical vein endothelial cells (HUVECs) were purchased from Lonza and were cultured in EGM (Lonza) and incubated at 37° C. under 5% CO₂. Human adipose-derived stem cells (hADSCs) were maintained in Dulbecco's Modified Eagle Medium media with 10% fetal bovine serum and incubated at 37° C. under 5% CO₂ and used before passage 3 in this study. Cell spheroids with a uniform size of 200 μm were fabricated using an agarose hydrogel microwell, as we previously reported. For three-dimensional (3D) culture of hADSC spheroids, the plate was coated with crosslinked hydrogels or NHCs and centrifuge at 300 g for 5 minutes [19]. Then, hADSC spheroids mixed with hydrogels or NHCs were seeded on top of pre-formed gels and cultured for 7 days.

### In Vitro Assessments

Cell viability was analyzed using Live/dead cell viability kit (Sigma Aldrich). For each cell type, cells were seeded (50 μL, 5×10⁶ cell/mL) in a 96-well plate coated with HA hydrogels or NHCs containing 150 μL medium and cultured for 1, 4 and 7 days. Cell adhesion assay was performed by similarly by seeding cell in pre-coated wells, and after 4, 24, and 48 hours, the cell numbers in the media suspension were measured to count the percentage of the unadhered cells. Cell migration and spreading of hADSCs in 2D and 3D cultures were examined by immunostaining. The cells were fixed with 4 w/v % paraformaldehyde for 10 minutes. And then stained with Phalloidin 568. Cell nuclei were counterstained with 4′,6-diamidino-2-phenylindole dihydrochloride (DAPI, Molecular Probes). All the images were acquired by LSM 780 Confocal Microscope.

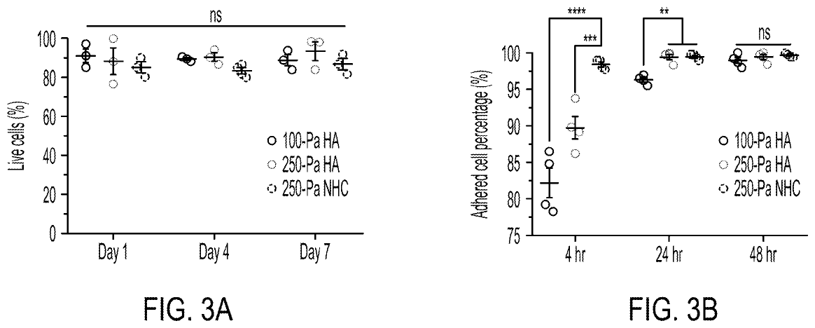

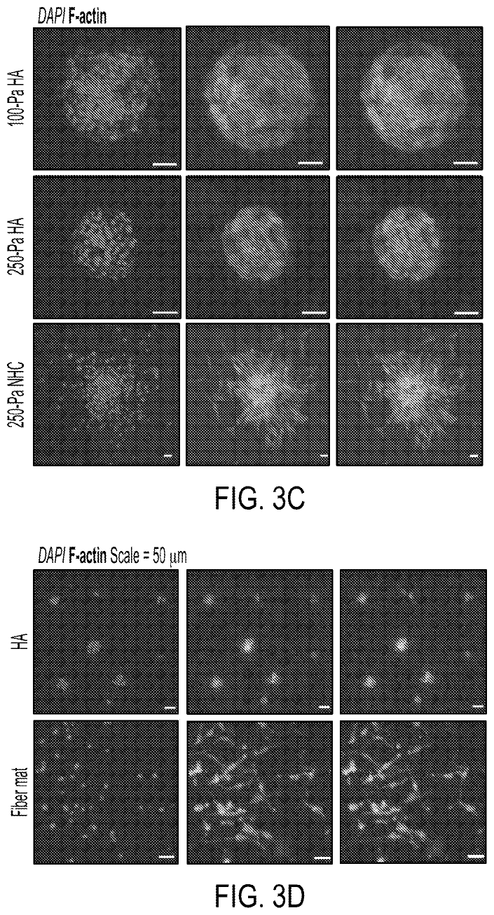

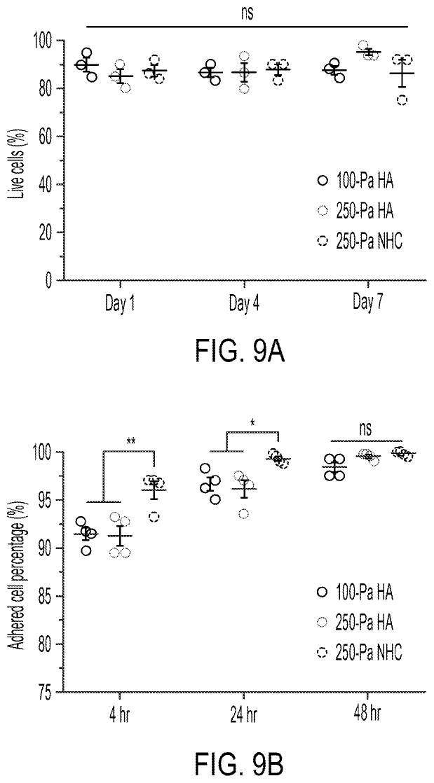

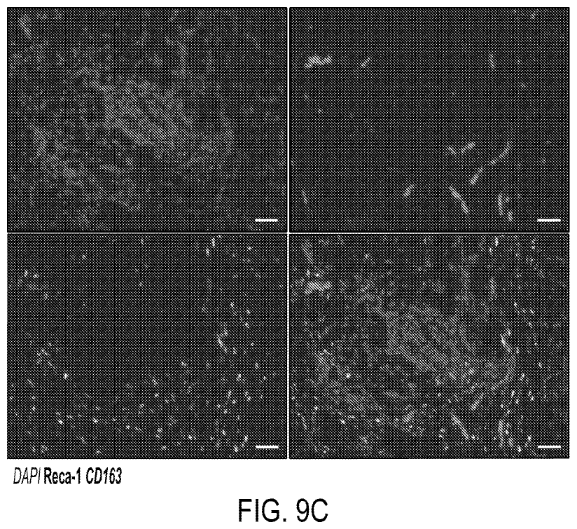

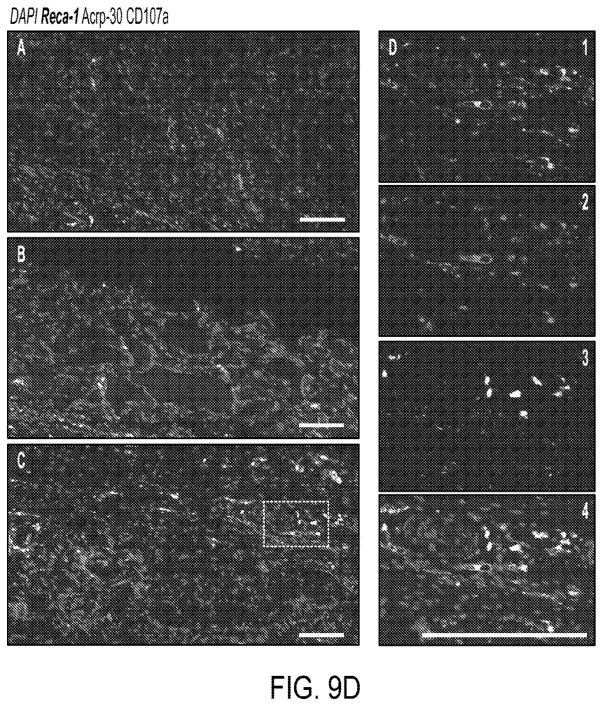

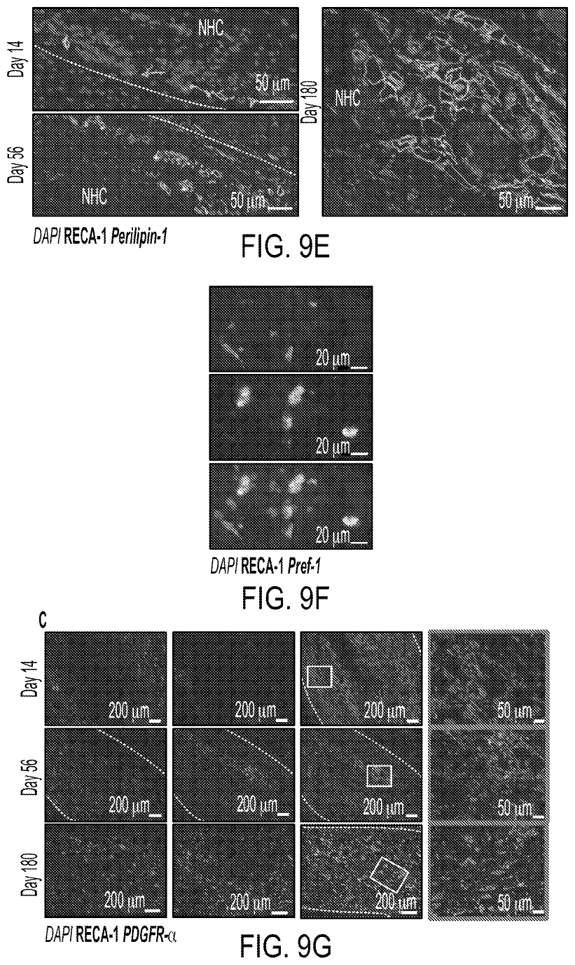

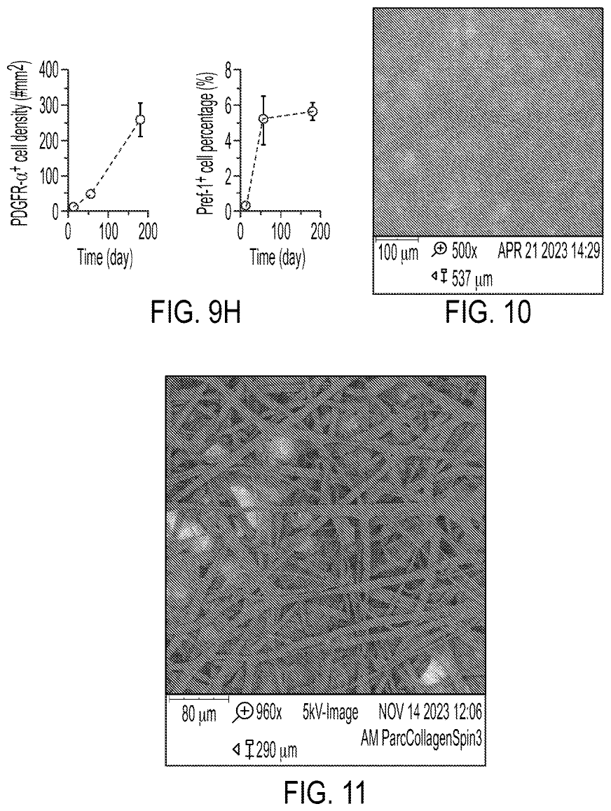

To further characterize the versatile abilities of this composite construct, we first conducted in vitro tests by culturing hADSCs and HUVECs on the three materials we prepared (100-Pa HA, 250-Pa HA and 250-Pa NHC with G₀′=100-Pa). Live/dead assay was utilized to assess the cell viability for the cells encapsulated in the three materials. It was observed that the cell viability for both the hADSCs and HUVECs were more than 90% for all three materials and did not show significant drops at day 4 and day 7 compared with day 1 (ns, P>0.05; FIG. 3A and FIG. 9A). We then examined the cell attachment in a 2D culture setup and observed that the 250-Pa NHC showed an improved cell attachment at 4-hour after cell seeding and the differences were minimized after 48 hours (FIG. 3B and FIG. 9B).

To evaluate the improvement of the cell migration by the nanofiber component, we firstly cultured the hADSCs on 100-Pa HA hydrogels and collagen fiber mat. As expected, a marked cell spreading behavior was observed for the cells cultured on fiber mat, and those cells cultured on HA hydrogels clustered and failed to migrate (FIG. 3D). Then, we examined the cell migration behavior in a 3D culture system where hADSC spheroids were encapsulated in 100-Pa HA control and 250-Pa HA (G₀′=100-Pa) for 7 days. In consequence, no noticeable cell migration was observed in 100-Pa HA control, which is in consistent with our previous studies showing the limited cell migration behaviors in HA hydrogels [23]. In contrast, cell spheroids in 250-Pa NHC showed a sprouting behavior consistent with the cell behaviors in 2D culture (FIG. 3C).

### Example 5: Subcutaneous Injection in Rats

To investigate the NHC-mediated macrophage polarization, angiogenesis and adipogenesis in vivo, NHCs and hydrogels were injected into the subcutaneous space of SD (Sprague-Dawley) rats (6-8 weeks old). Three replicates were performed for each formulation at a volume of 200 L/injection. The rats were sacrificed at 7, 14, and 56 days after injection. The explant sizes were measured by calipers to get a rough geometry of the retained volume. The injection explants were then immediately reserved for immunohistochemistry in 4 w/v % paraformaldehyde (PFA) for 3 days after sacrificing the animals. The explants were then embedded in paraffin after a series of dehydration, following by the sectioning to get slices within 10 μm. For histological staining, the tissue slices embedded in paraffin slices were deparaffinized and then stained with hematoxylin and eosin (H/E staining) and Masson's trichrome staining for histological analysis. For quantitative analysis, three slices were stained for each injection. The cellular infiltration area in the histology images was determined in ImageJ by selecting the implanted area, converting to grayscale, and measuring the dark area (the original implant is lighter than the surrounding tissue, allowing for simple thresholding).

For immunofluorescent staining, the tissue slices embedded in paraffin slices were deparaffinized and rehydrated to activate the surface for immunostaining. Briefly, explants were immersed in xylene, 100% ethanol, 95% ethanol and 70% ethanol for 10 minutes, respectively, and then placed in IC buffer inside boiling pot for 20 minutes to activate the tissue surface. Sections then were permeabilized with 0.5% Triton X-100 solution and blocked by 4% donkey serum in PBS for 2 hours. The samples then were incubated with primary antibodies (Table 1) overnight at 4° C. Cy3 and Cy5 affinity secondary antibodies (Jackson ImmunoResearch Laboratories) including Cy3 conjugated donkey anti-mouse (Cat. #715-165-151), Cy3 conjugated donkey anti-goat (Cat. #705-165-147), Cy5 conjugated donkey anti-rabbit (Cat. #711-175-152) were applied to the sections at room temperature for 2 hours. Sections were then washed three times with PBS, and counterstained with DAPI for 15 minutes. Six random fields of each specimen were imaged by ZEISS LSM 780 Confocal Microscope for quantitative analysis.

TABLE 1
Primary antibodies used in this study.

| Antibody | Company | Catalog Number |
|---|---|---|
| CD68 | Abcam | ab31630 |
| CD38 | GeneTex | GTX37752 |
| CD163 | Abcam | ab182422 |
| αSMA | Abcam | ab5694 |
| Reca-1 | Abcam | ab9774 |
| Acrp-30 | Bio-Techne | AF3100 |
| Perilipin-1 | Abcam | Ab3526 |
| CD107a | Novus | NBP1-86557 |

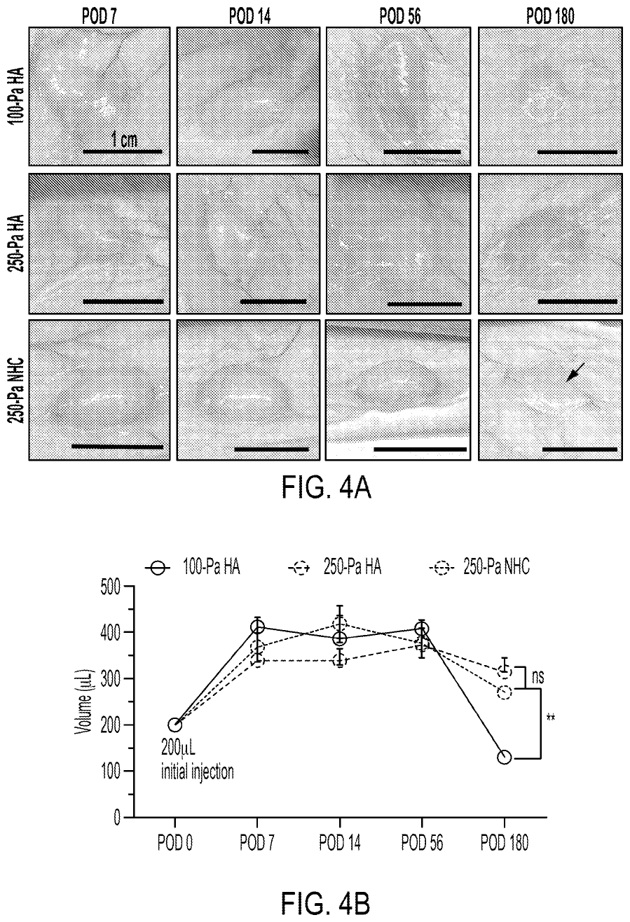

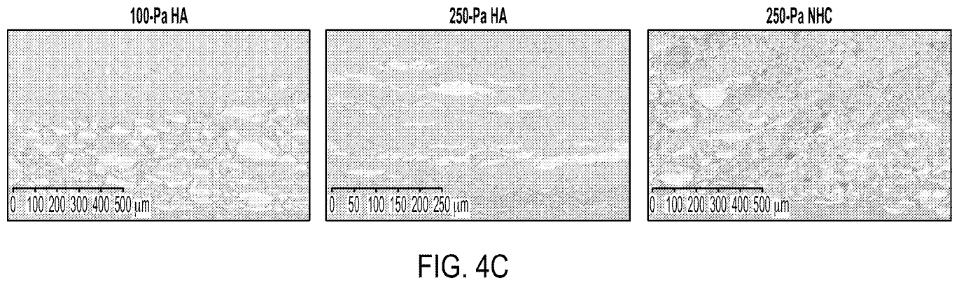

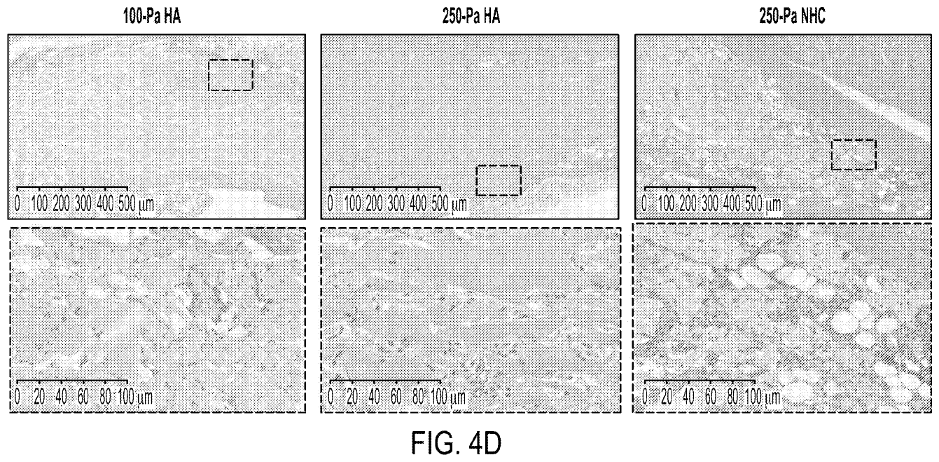

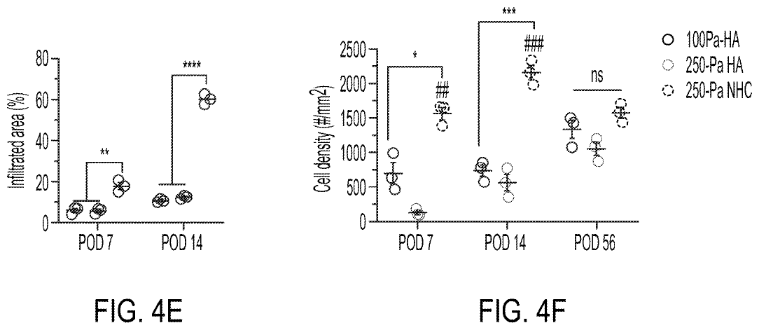

To investigate the bio-stimulatory effect mediated by the collagen fiber components in vivo, we subcutaneously injected 200 μL of the NHC (G′=250-Pa and G₀′=100-Pa) and two hydrogel controls (G′=100-Pa and 250-Pa) into the back area of SD rats. The shape retention of the materials was measured by gross images after sacrificing rats at POD 7, 14 and 56. The measurements indicated that three groups of materials underwent an initial swelling and kept at around 300-400 μL volume until POD 56, then a small decline in volume for the 250-Pa NHC and 250-Pa HA control (with volumes still greater than the initial injection volume), but a large decline in the 100-Pa HA control group (FIGS. 4A and 4B). Moreover, an elevated cell infiltration was found in NHC at POD 7 in the histology images (FIG. 4C)) with around 17% of cell infiltration area compared with around 6% at two hydrogel controls (P<0.01), and the infiltration of the cells kept increasing in the NHC at POD 14 with around 60% of the areas covered by infiltrated cells, which was significantly higher than those of hydrogel controls (P<0.0001; FIGS. 4E and 4F). The cell density was also measured based on the H&E staining images, which showed a consistent trend at POD 7 (P<0.05) and POD 14 (P<0.01) when comparing the infiltrated cells in NHC with those in hydrogel controls (FIGS. 4E and 4F). The continuously increasing cell infiltration inside the retained NHC at POD 7 and POD 14 suggests that those infiltrated cells may be recruited by the presence of bio-stimulatory collagen fibers and this effect maintained due to the good shape retention of the materials.

At POD 56, the H/E images and the cell density analysis revealed an enhanced angiogenesis and adipogenesis in parallel with some neo-tissue formation (FIG. 4D). The cell density analysis indicated that the cell numbers inside 250-Pa NHC plateaued around 1500 cells/mm² and showed no significant difference from the 100-Pa HA control (ns, P>0.05; FIG. 4F). While not being bound by theory, this may indicate the 250-Pa NHC injected area was already undergoing tissue remodeling and thus fewer additional cells were infiltrating into the NHC.

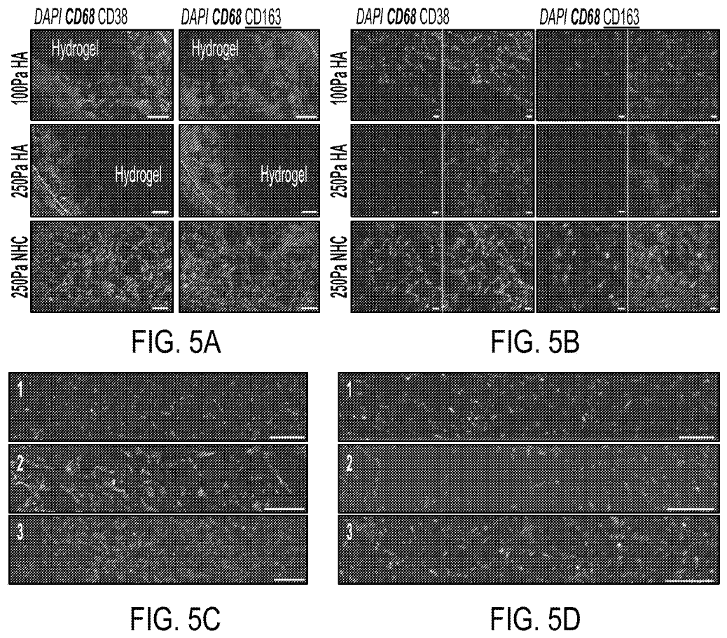

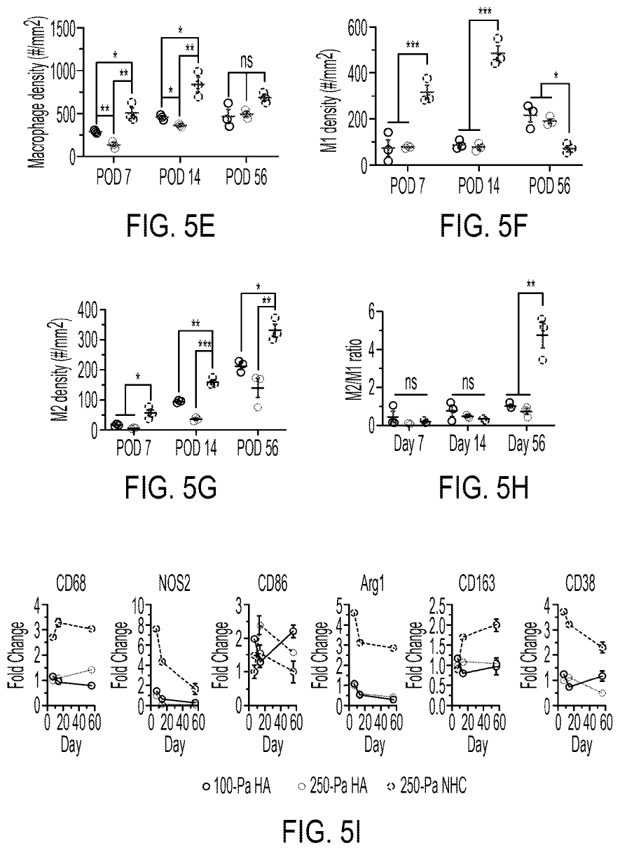

To understand how the bio-stimulatory effect of the NHC could lead to an elevated tissue remodeling process, we examined whether this response was correlated to the macrophage infiltration and polarization. The activated macrophages presented two phenotypes, pro-inflammatory M1 and pro-regenerative M2. Three markers, CD68, CD38 and CD163 were harnessed in our study to identify the pan-macrophages, M1 macrophages, and M2 macrophages, respectively [24-26]. At POD 7, 250-NHC were able to recruit around 2 folds of CD68⁺ pan-macrophages compared with 100-Pa HA (P<0.05) and 250-Pa HA (P<0.01) (FIGS. 5A and 5E). Similarly, both CD38⁺ M1 macrophage (P<0.001) and CD163⁺M2 macrophages (P<0.05) started to be activated at a higher level in NHC and was confirmed by the statistical analysis (FIGS. 5B, 5F and 5G). At POD 14, similar trends were observed for CD68⁺ and CD38⁺ cells, but the CD163⁺ M2 macrophage expression in 250-Pa NHC was noticed to be more augmented compared with 100-Pa HA (P<0.01) and 250-Pa HA (P<0.001) controls (FIGS. 5B, 5D and 5G). At POD 56, consistent with the H/E histological results, the number of infiltrated CD68⁺ pan-macrophages in three groups did not show significant difference (FIG. 5E), suggesting that the enhanced recruitment of the macrophages in the NHC group seen at earlier timepoints was halted by this timepoint. Moreover, a significantly lowered CD38⁺ M1 macrophage density in NHC at 72.9±19.8 cells/mm² was noticed, while the two hydrogel controls were at 216.0±52.0 and 192.0±17.8 cells/mm² respectively (P<0.05; FIGS. 5E and 5G). Furthermore, this decreased expression of M1 phenotype in 250-Pa NHC came with an increased expression of CD163±M2 macrophages in NHC at 331.0±36.6 cell/mm² as compared with 100-Pa HA control at 213.6±19.4 cells/mm² (P<0.05; FIGS. 5F and 5G). The improved macrophage infiltration at POD 7 and the skewed polarization with higher M2/M1 ratio at POD 56 in NHC (FIG. 5H) suggest that the porous hydrogel network (seen in FIG. 1E) reinforced by bio-adhesive collagen nanofibers may accelerate the polarization of the recruited macrophages towards the pro-regenerative phenotype, and thus quickening the tissue remodeling observed in histological images (FIG. 4D).

The pattern of early regenerative inflammation induced by NHC was confirmed by quantitative gene expression analysis. Pan-macrophage related gene CD68 was upregulated more in the NHCs on Day 7, 14 and 56 compared to the HA controls, indicating a continuous bio-stimulatory effect for macrophage recruitment (FIG. 5I). Further, M1-specific genes NOS1 and CD38, showed early elevation by 5-fold in NHC matrix compared with HA controls on Day 7, with decrease thereafter (FIG. 5I). By contrast, M2-specific genes Arg1 and CD163 genes were expressed early and persisted through the intermediate timepoints in NHC matrix (FIG. 5I).

### Example 6: Bio-Stimulatory NHC Mediated Endothelial Infiltration and Angiogenesis

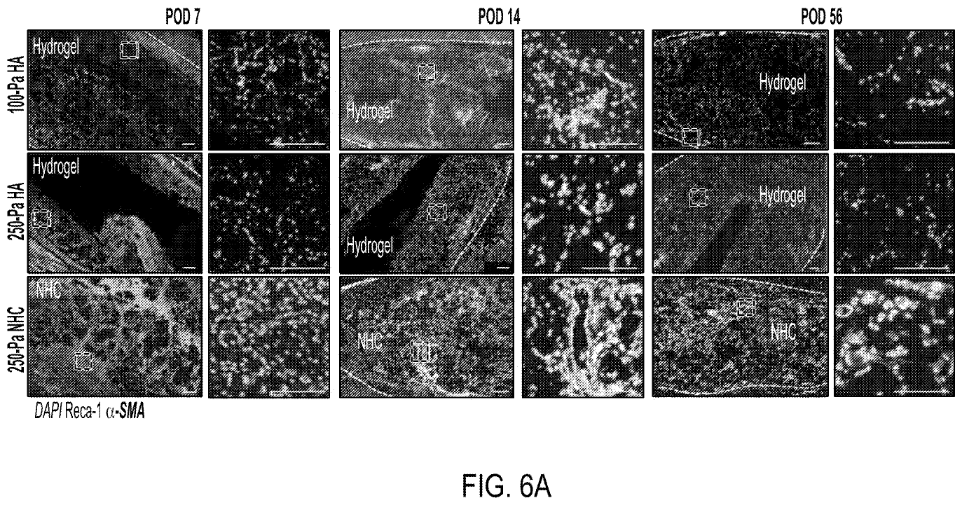

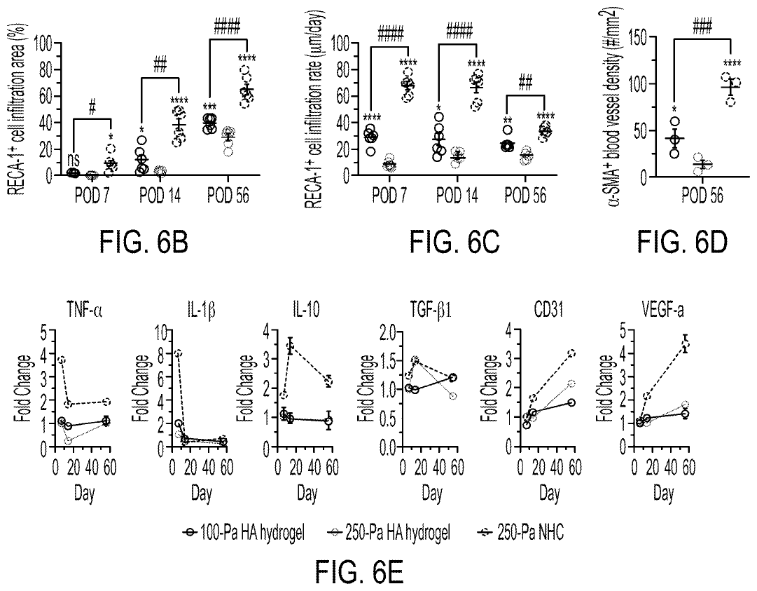

To investigate the blood vessel formation observed in histological images, angiogenesis, as an aspect of tissue remodeling, was investigated in vivo. The infiltrating area and rate of host vascular endothelial cells (RECA-1⁺) were analyzed using immunofluorescence [27]. At POD 7, the NHC group showed the highest endothelial cell infiltrating area 250-Pa NHC: 9.2±6.1%, 100-Pa HA: 1.7±0.6% (P<0.05), and 250-Pa HA: 0.2±0.1% (P<0.05); FIGS. 6A and 6B. The infiltrating rate of the endothelial cells in the NHC group was also accelerated at 67.5±7.2 m/day, which was significantly faster than that of 100-Pa HA (28.8±5.9, P<0.0001) and 250-Pa HA (8.7±2.7, P<0.0001) (FIGS. 6A and 6C). At POD 14, a similar trend was observed in terms of the endothelial cell infiltration rate and area, and as the infiltrating rate in NHC kept at a high level of 66.5±10.4 μm/day, the endothelial cell started to spread broadly within the NHC (FIGS. 6A and 6C).

At POD 56, well-formed blood vessels were studied by investigating α-SMA⁺ vascular structures in addition to the RECA-1⁺ cells [28]. As expected, the α-SMA⁺ blood vessel density inside the NHC was notably higher than the 100-Pa hydrogel (P<0.001) and the 250-Pa hydrogel (P<0.0001) (FIG. 6D). Even though the endothelial cell infiltrating rate was decreased to 33.6±3.7 μm/day in NHC, which may be because the host endothelial cells were already distributed in the whole NHC construct, the infiltrating area increased to 64.9±9.6% compared with relative low area in hydrogel controls (P<0.0001) (FIGS. 6A, 6B and 6C).

To understand if the programmed macrophage polarization altered the local inflammatory behavior in a gene expression level, a few pro-inflammatory genes (TNF-α, IL-1β), and pro-regenerative genes (IL-10 and TGF-01) were measured using qPCR. The qPCR results revealed that the two pro-inflammatory genes were up-regulated (3.7-fold and 7.9-fold, respectively) in the early day 7 timepoint, and then down-regulated to low level (1.7-fold and 0.4-fold, respectively) in the NHC matrix (FIG. 6E). Meanwhile, two anti-inflammatory genes, IL-10 and TGF-01, were selected to measure the regenerative condition. Like the observations in macrophage polarization, IL-10 was up-regulated in NHC matrix, indicating the onset of regenerative behaviors in NHC matrix, but the measurements of TGF-β1 did not show significant differences among three groups (FIG. 6E).

The host endothelial cell ingrowth in the bio-stimulatory NHC corresponds to the enhanced cell infiltration observed in histological images and the enhanced macrophage recruitment and conditioning. To understand the correlation between angiogenic response and the skewed polarization of macrophages inside the NHC, we further investigated the immunofluorescence of RECA-1⁺ and CD163⁺ cells at POD 14, and the results showed that an ample number of CD163 M2 macrophages were localized close to the endothelial cell clusters, suggesting the early vasculature formation may be enhanced by the pro-regenerative macrophages (FIG. 9C).

### Example 7: Enhanced Adipose Tissue Formation and its Correlation with the Recruited

### Progenitor Cells

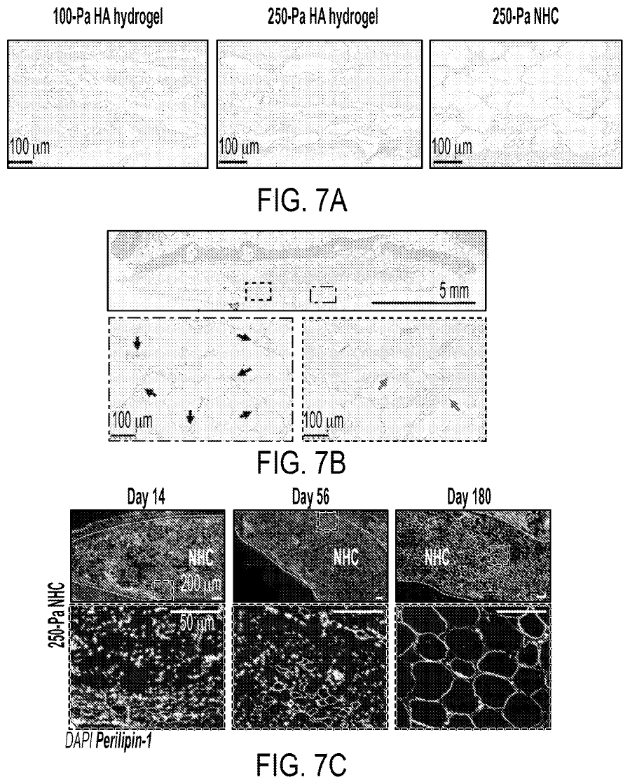

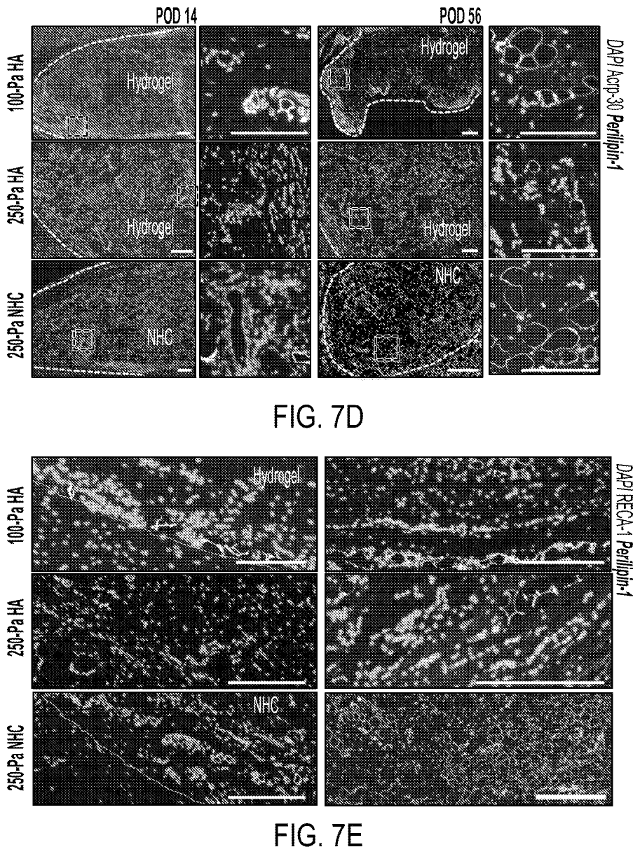

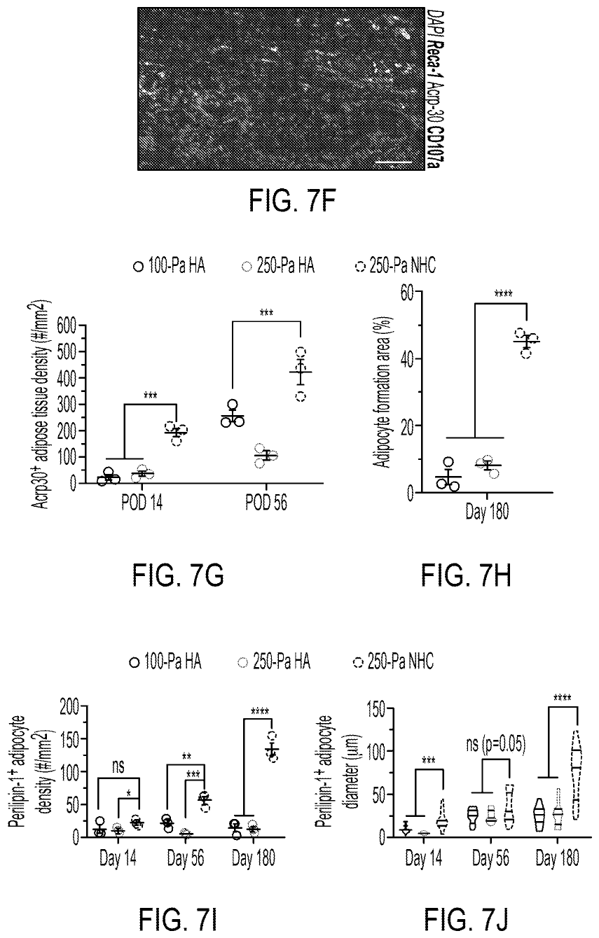

Our previous work suggested that after injecting NHC in vivo for 28 days, adipose tissue started to form inside the NHC [19]. At the 180-day timepoint, this trend has continued to the point where the implanted NHC gel has substantially transformed into vascularized adipose tissue, while the HA-gel controls have not (FIG. 7A). The resulting mixture of adipose tissue and remaining NHC gel maintains the volumization at the implant site, has numerous functional blood vessels, and has tight integration with the remaining implanted gel, without fibrosis (FIG. 7B). The progressive transformation into adipose tissue is verified with immunofluorescence, with Perilipin staining showing the increase in mature, lipid-bearing adipocytes over time in the gel (FIG. 7C).

The improved adipose tissue formation inside NHC indicate an accelerated tissue remodeling process facilitated by the bio-stimulatory NHC. To investigate the mechanism how the adipose tissue was forming inside NHC, we used immunofluorescence to mark the associations between cell types over time. Firstly, we co-stained the adipose tissue marker adiponectin (Acrp-30) with Perilipin-1 (to mark mature fat formation) inside the NHCs. Results showed that Acrp-30 expression was also elevated inside NHCs compared to HA controls on Day 14, when the Acrp-30⁺ adipocytes had already infiltrated at a density around 160 cells/mm² inside the composite, a rate about five-fold higher than seen in the fiber-less HA controls (P<0.001), and trend which continued at Day 56 (FIG. 7D). The fat formation stained by perilipin-1 indicated a similar trend (P<0.0001) where numerous fat particles were forming at the periphery of the injected 250-Pa NHC. At POD 56, the fat particles started to be present at the periphery of 100-Pa HA control, while an ample number of those fat particles were distributed inside the NHC (P<0.0001 (FIGS. 7D and 7I). However, the density of adipocytes continued to increase in NHC with time, and by day 180 the density of adipocytes reached 134.5±14.0 cells/mm² to five-fold higher than either hydrogel control (p<0.0001; FIG. 7H). Not only were there more adipocytes, but they were significantly larger with mature lipid droplets in the NHC at Day 180 (FIG. 7J). These findings suggest that NHC not only supports the formation of adipocytes, but also favors the microenvironment for adipocytes growing and distribution. Interestingly, Acrp-30 can be expressed on endothelial cells as well as adipocytes, and some vascular structures were observed very close to adipocytes in the remodeling NHC (FIG. 7B). These observations led us to hypothesize that the adipocyte formation may have some correlation to the infiltration and formation of blood vessels.

To further investigate this correlation, the immunofluorescence of Reca-1+ endothelial cells and Perilipin-1+ adipocytes was performed. A close localization of endothelial cells and adipocytes were observed at POD 14 at the periphery of 100-Pa HA and 250-Pa NHC, and widely across the 250-Pa NHC at POD 56 (FIG. 7E). Moreover, some Acrp-30+ adipocyte signals were even mingled with RECA-1+ host endothelial cells, indicating a strong correlation between the adipogenesis and angiogenesis. Previous studies showed that vascular progenitor cells had the potential to differentiate into adipocytes to facilitate the tissue remodeling [31, 32]. We then added the CD107a marker to identify the infiltrating perivascular progenitor cells. The POD 14 results revealed that an elevated CD107a⁺ pericytes infiltration in 250-Pa NHC, and the signals of RECA-1, Acrp-30 and CD107a were closely localized, indicating the progenitor cells recruited by the bio-stimulatory NHC may facilitate the angiogenic and adipogenic responses (FIGS. 7F and 9D).

As noted, we observed a close localization of endothelial cells and adipocytes on Day 14, Day 56, and Day 180 inside the 250-Pa NHC (FIG. 9E). Then, a co-staining of Pref-1+ pre-adipocytes and Reca-1+ endothelial cells was performed to understand the correlation between vasculature formation and differentiation or the origin of adipocytes. According to confocal images, it was noticed that Pref-1+ pre-adipocytes also localized in proximity to endothelial cells (FIG. 9F). In addition, the quantitative analysis showed that on Day 14, almost no Pref-1+ cells were observed inside NHC. However, on Day 56, around 5% of cells started to express the pre-adipocyte marker and this percentage remained consistent until Day 180 (FIG. 9H). These observations suggest that there is a low population of cells starting to differentiate into adipocytes, while the origin of these adipocytes remains unknown.

A number of recent studies have identified perivascular cell populations, particularly PDGFRα+ cells, as adipocyte stem cells capable of differentiating into preadipocytes and finally mature adipocytes. Strikingly, immunofluorescence images showed that the number of PDGFRα+ cells continued to increase from Day 14 to Day 180 in NHC, and they were closely localized to the endothelial cells (FIGS. 9G and 9H). These results coupled with the kinetics of tissue remodeling suggest a self-sufficient mechanism for NHC to generate adipose tissue in vivo. Specifically, NHC recruits and polarizes macrophages in a pro-regenerative direction, leading to angiogenesis and recruitment of adipocyte precursors in the perivascular space.

### Example 8: Concentration of HA is Critical for Successful Gelation

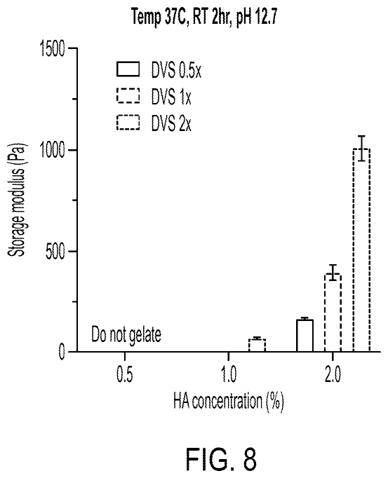

The gelation system can be dependent upon the HA and DVS concentrations. When the HA concentration was at 0.5 w/v % (5 mg/mL), the solution did not gel, regardless of the crosslinker concentration. At 1.0 w/v %, the solution did not gel at low crosslinker concentration, and only gelled very weakly at high crosslinker concentrations, while the solution gelled robustly at a HA concentration of 2 w/v % (20 mg/mL) (w/v % is percent weight based upon the weight of the component in grams per 100 mL of solution and 2 wt % also can be expressed as 20 mg/mL. The weight per volume is equivalent to the weight per weight (w/w %) if the composition has a density of 1 g/mL, which is approximately the density of these examples) (FIG. 8).

### Example 9: Pre-Swelling Step is Crucial to the Successful Sterilization of the Composite by Autoclave

The dialysis and pH neutralization steps are necessary for achieving the thermal stability needed for autoclaving. The fiber-hydrogel composite was generated using a formulation that generates robust gels, with 2 w/v % HA, 1×DVS, 37° C., pH 13, 3 w/v % fiber loading composite. However, the gelled samples were then autoclaved but without dialysis. After autoclaving, the resultant NHC was liquified.

### Example 10: Alternative Crosslinker: BDDE-Crosslinked Composite Instead of DVS-Crosslinked Composite

To evaluate whether the fiber hydrogel composite could be produced with an alternative crosslinker that is used in many commercial products, we attempted to make gels with BDDE instead of DVS. Using the same reaction conditions of the previously described successful DVS-crosslinked gels, however, we were not able to produce stable crosslinked gels with similar concentrations of HA as with the DVS groups. In those DVS-crosslinked groups, a HA concentration of 2.0 w/v % was required to form robust gels. However, groups crosslinked with HA concentrations of, 2, 4, 6, and 8 w/v % (all mixed with 1 v/v % of BDDE at pH 13 (0.1 M NaOH solution) reacted at 40° C.) did not form a gel. A group with 10 w/v % of HA was able to form a robust gel after 16 hours of reaction time, forming a 300-Pa gel. This is a 5-fold higher concentration of HA than needed for the DVS crosslinking.

### Example 11: Fiber-Hydrogel Composites with Recombinant Collagen Samples Electrospinning and Crosslinking of Demulcent SFA

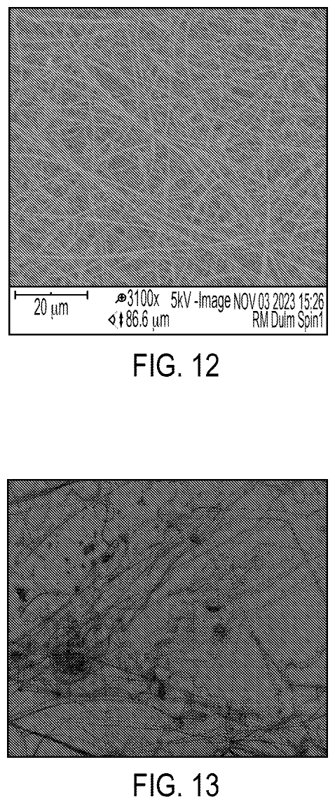

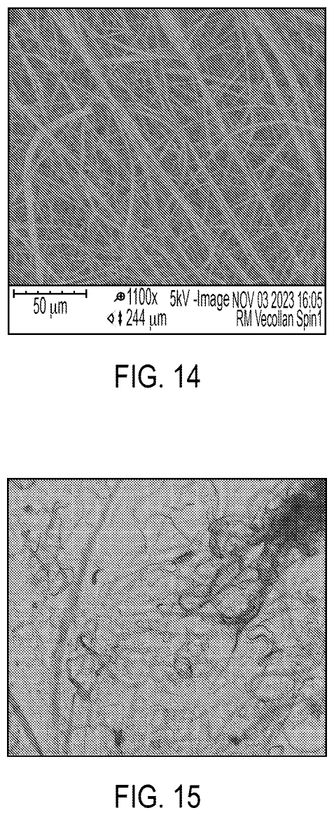

Similar to the processed used in generating electrospun porcine type 1 atelocollagen (Nitta) (FIG. 10) and porcine gelatin (Sigma) (FIG. 11), which have been used for preparation of fiber-hydrogel composites, we also generated electrospun collagen-fibers using recombinant collagen Vecollan (Evonik) (FIG. 12) or recombinant collagen Demulcent SFA (Jland Biotech) (FIG. 14), and effective crosslinked these fibers as shown in the optical microscope micrographs of electrospun fibers following staining with picro Sirius red dye (FIGS. 13 and 15). These collagen fibers are stable in aqueous environments and can be formed into composites with HA and retain fibrous morphology after gelation and autoclave sterilization.

As an example, a Demulcent SFA (human recombinant collagen; Jland Biotech) collagen solution at a concentration of 13.2 w/w % dissolved in in 2,2,2-trifluoroethanol is extruded from 5-mL syringe through a 27-G blunt needle at a rate of 2.0 mL/hour. Collagen fibers are collected on a drum rotating at approximately 140 rpm with an electric potential of 15 kV on the needle tip and a −4.5 kV on the collector. Fibers are treated with 1 v/v % DVS in ethanol for 24 hours, then 0.7 w/v % glutaraldehyde in ethanol for 20 hours, respectively.

Both DVS and glutaraldehyde can crosslink proteins through the amine groups on the polypeptide chains. The amine group of the N-terminus of the peptide and surface lysines of the protein are targeted. However, using DVS or glutaraldehyde alone wouldn't crosslink Vecollan or Demulcent SFA efficiently. Electrospun recombinant protein fibers are crosslinked via 2-step process. The resulting fibers have a limited swelling in water and in gel composite.

### Fiber Swelling Test

Fibers are suspended in water and stained by picro sirius red, and a minimal swelling is observed, indicating effective crosslinking of the collagen fibers (FIG. 15).

### Hydrogel Composites Preparation

Hydrogel composites are prepared by mixing 2.08 g of HA stock solution with 0.42 mL water and 15 mg of crosslinked collagen fibers, followed by adding 50 UL of 5 M NaOH and 66 L of DVS. The mixtures are incubated at 30° C. for 2 hours. The reaction is neutralized with 20 mM of NaH₂PO₄ solution. Gel composites are dialyzed against PBS then spun down at 2500×g, then autoclaved with liquid cycle for 30 min at 121° C.

### Rheometry Tested Per Example 3.

| Collagen type | Swelling | | G′ at 10% | G″ |
|---|---|---|---|---|
| for fibers | ratio | Autoclave | strain (Pa) | (Pa) |
| Vecollan | 2.6 | − | 170 | 37 |
| Vecollan | | + | 72 | 14 |
| Demulcent | 2.6 | − | 218 | 32 |
| Demulcent | | + | 109 | 23 |

### DISCUSSION

We disclose herein, inter alia, a mechanically reinforced nanofiber-hydrogel composite (NHC) that not only serves as a soft tissue substitute, but also incorporates a bio-stimulatory collagen nanofibers to promote the host cell infiltration, macrophage conditioning, and the final adipose tissue restoration. The interfacial bonding between the HA network and collagen nanofibers provides an effective network to retain collagen fibers inside the composite and enhances the composite stiffness with relatively low fiber loading density. Our results indicate that the present materials and compositions (including NHC gel) can attract more host cell infiltration than the HA controls, condition the infiltrating macrophages, escalate the angiogenic responses and most importantly accelerate the adipose formation. Additionally, the feasibility of terminal sterilization, compatibility with recombinant collagen, and excellent injectability of the NHC represent the translatability of the materials.

Previous studies investigated the impact of storage modulus (G′) for hyaluronic acid (HA) hydrogels on host cell infiltration and the shape retention, but those studies used five- to ten-fold lower levels of gel stiffness compared to the NHC used in this example [33, 34]. Our previous work showed that a higher storage modulus can significantly impede the host cell infiltration and neo-vascularization [19]. Therefore, we produced a NHC sample with G′=250 Pa (G₀′=100 Pa), with two HA hydrogel controls at G′=100 Pa and G′=250 Pa. When comparing the two HA controls, it was noticed that 250-Pa hydrogel could hardly allow the host cell infiltration at POD 7 and POD 14, and the POD 56 angiogenesis and adipogenesis were also minimal. The 100-Pa HA hydrogel, even though showed a relatively higher cell density compared with the 250-Pa HA hydrogel at POD 56, its storage modulus does not lie in the range of most soft tissues (150- to 500-Pa), and fails to mimic the host tissue structure [35]. The 250-Pa NHC has a similar crosslinking density to the 100-Pa HA hydrogel, but with 3 w/v % of collagen nanofiber loading, the storage modulus is reinforced to 250-Pa, which lies in the 150- to 500-Pa range, indicating it could mechanically be a soft tissue substitute. Moreover, the results show that the bio-stimulatory collagen fibers recruited ample host cells (around 2.5-fold compared to 100-Pa HA hydrogel in density) to the NHC, and the infiltrating cells were distributed evenly at POD 14, indicating that the gel remained cell permissive and isotropic with even fiber dispersion (anisotropy or uneven fiber loading is expected to bias the cell response to be more clustered in a stiffer, fiber-rich region).

As the NHC recruited abundant host cells, we noticed that a large number of macrophages were also attracted to the NHC materials. Around 1.5-fold of CD68⁺ pan-macrophages were attracted to the NHC at POD 7 compared to the 100-Pa HA control, and this ratio climbed to around 2-fold at POD 14, suggesting the prolonged bio-stimulatory effect of the collagen nanofibers inside NHC. Our previous work showed that PCL nanofibers could shift the macrophage polarization towards M2 [19]. In this example, it was observed that collagen nanofibers may also conditioned the macrophages towards a M2 phenotype as the M2/M1 ratio in NHC group was found around 2-fold to those in HA controls at POD 56. Moreover, the decrease of M1 phenotypes in NHC group at POD 56 compared to those at POD 14 suggests that those M1 phenotypes may re-polarized to M2 phenotypes inside the NHC materials. Alternatively, this M2 polarization trend could be explained if the M1 cells present at the implant site were to preferentially migrate away from the implant site, while incoming naïve macrophages are preferentially polarized to M2 phenotypes.

Angiogenesis is considered limited in hyaluronic acid (HA) hydrogels [19, 36]. With NHC, endothelial cell infiltration rate was markedly promoted to around 2.5-fold in the NHC compared with HA controls. Also, the α-SMA⁺ cells were also around 2.5-fold in NHC, suggesting the accelerated maturation of the newly formed blood vessels. Moreover, we also found a close localization of endothelial cells and CD 163⁺ M2 macrophages, which indicates that the neo-vasculature formation may be facilitated by the skewed M2 macrophage polarization. Therefore, it is likely that the bio-stimulatory collagen nanofibers recruited macrophages and conditioned them to M2 phenotypes, then promoting the neo-vasculature formation in NHC.

Generating soft tissue with the hydrogels are challenging because the spontaneous host cell infiltration and remodeling is a slow process [37, 38]. In the present example, by incorporating collagen nanofibers, which was shown to promote adipogenesis for progenitor cells in vitro, accelerated the adipogenesis in vivo [39]. At POD 14, some adipocytes started to form at the periphery of the NHC materials, and the adipocytes became more abundant and distributed at POD 56 in NHC, and vascular adipose tissue accounted for almost half of the implant area by POD 180. Furthermore, it was found that those adipocytes were localized closely to the endothelial cells. It is likely that the early adipose tissue formation needs the blood supply, and thus the vasculature structures form closely to those adipocytes. Also, we have found that progenitor cells such as CD107a⁺ progenitor cells, may promote adipogenesis by direct differentiating to adipocytes. Here, with the immunofluorescence images, we observed a similar trend that more CD107a⁺ cells were recruited to the NHC, and they share a close location to the RECA-1⁺ endothelial cells and Acrp-30⁺ adipocytes.

This improvement is possible by how the synthetic yet biomimetic composite or gel induces macrophage recruitment and polarization, resulting in a controllable inflammatory response. Inflammation is a crucial tissue response for injury, infection, and tissue loss to facilitate homeostasis. Depending on the local host tissue microenvironment and the degree of inflammation, the outcome normally comes with imperfect fibrotic tissue formation or scarring. Considering the nature of inflammation to achieve reparative outcome, recent studies indicate that a programmed inflammatory response may optimize the regenerative responses in different tissues. This is achieved by programming the macrophages, which are the bridges between inflammation and remodeling. While macrophages also have a central role in the chronic inflammation development, by altering the polarization of macrophages, they can be programmed to facilitate the pro-regenerative outcome.

In our work, by introducing biostimulatory collagen NHC matrix subcutaneously, we were able to create a sustained and mild inflammatory microenvironment with continuous host macrophage recruitment. Strikingly, instead of deteriorating towards chronic inflammation and fibrotic tissue formation, a pro-regenerative phenomenon was observed in the NHC matrix. Pro-regenerative CD163+ macrophages were more polarized in NHC matrix compared to the HA controls. Clearly, the bio-stimulatory nature of the NHC matrix altered the trajectory of the local inflammatory responses. Following the macrophage polarization, reparative genes such as IL-10 and VEGF-a were upregulated in NHCs, leading to a more angiogenic behavior and soft tissue formation when the study was performed for 6 months.

There are several significances of this study. First, the NHC elicits excellent angiogenesis and soft tissue restoration without introducing any exogenous cytokines or cells. Comparing with most existing studies that focus on repair-supporting biologics, this NHC material itself can produce a long-lasting repair effect. Moreover, in preferred aspects, the present composites can provide for physical mixing or conjugation of exogenous growth factors or cytokines, which may furthermore improve the repair outcomes.

Another significance of these examples is its investigation of the correlation among the polarization of recruited macrophages, the induced angiogenesis, the escalated adipose tissue formation, and the infiltration of progenitor cells.

A final significant aspect is the translatability of this material as the terminal sterilization using autoclave does not significantly change its mechanical behaviors. Also, the safety record of HA, collagen and DVS in clinical uses is also favoring the future clinical translation of the NHC material. The compatibility with alternative collagens such as recombinantly sourced collagen allows for fully synthetic products that further improve the translatability of the invention, overcoming religious, lot-to-lot variability, and allergy issues intrinsic to animal-derived products.

In conclusion, we have developed a granular collagen nanofiber-reinforced hydrogel composite those functions as a soft tissue substitute by mimicking the mechanical properties and microarchitecture of the native tissue. The porous structure of the NHC and the bio-stimulatory collagen fibers can facilitate host cell infiltration. The NHC can further program the local inflammation by polarizing the infiltrated macrophages towards a pro-regenerative M2-like phenotype and thus continuously promotes angiogenesis and neo-vasculature ingrowth, leading to the formation of a vascularized adipose-like tissue in vivo. This adipogenic outcome highly correlates with neo-vasculature in the remodeling matrix, suggesting PDGFRα+ perivascular cells as a potential source of adipocyte progenitors. The NHC-programmed regenerative inflammation represents a novel mechanism enabled by our injectable biostimulatory matrix design and may provide broader implications in soft tissue reconstruction and many other regenerative therapies. In preferred aspects, the present compositions and composites can provide soft tissue augmentation and restoration with a shelf-stable, off-the-shelf gel (composite) without requiring the incorporation of cells or growth factors.

In preferred aspects, beads used in materials and compositions are irregular, non-spherical gel bead morphology with evenly dispersed (with random orientations as opposed to aligned or planar orientations) fibers (particles with aspect ratios significantly greater than 1.0). The fibers decorate the gel bead surface and can be made to produce an isotropic gel. The gel can be made into various forms including injectables for aesthetic or therapeutic use. The composites preferably have high thermal stability, enabling the use of steam autoclaving for terminal sterilization and resulting in a long shelf life at ambient temperatures.

In certain aspects, mechanical and biostimulatory properties of the present composites can be dependent upon the high aspect ratio of the fibrous component, which are distinct from other particulate forms which have low aspect ratios such as spheres.

As referred to herein, the aspect ratio is the particle length divided by its width. Preferred fibers of the present compositions and composites are approximately up to or about 0.6, 0.7 or 0.8 μm in diameter or cross-section dimension, or from about 0.4 or 0.5 μm to 0.8 or 0.9 μm in diameter or cross-section dimension, or from 0.5 or 0.6 μm to 0.7 or 0.8 μm in diameter or cross-section dimension, even the shortest fiber fragments (3 μm long) would have an aspect ratio of at least 5, with longer fibers (100 μm and above) having an aspect ratio of 166 or greater.

The present composite material with a mean aspect ratio of at least 20 (corresponding to fibers 20 μm long, 1 μm in diameter) are expected to result in improved mechanical and biological responses compared to gels with aspect ratios under 20 (shorter and/or wider fiber fragments). In one evaluation, the composite materials with the highest mechanical reinforcement with fibers of 40 to 100 μm in length) had aspect ratios of the fibers from about 60 to about 170.

### REFERENCES

1. Sclafani, A. P. and S. Fagien, Treatment of injectable soft tissue filler complications. Dermatologic Surgery, 2009. 35: p. 1672-1680.
2. Takeuchi, A., et al., Occlusive dressing for large soft tissue defects following soft tissue tumor excision. Journal of Orthopaedic Science, 2009. 14(4): p. 385-390.
3. Kai, D., et al., Mechanical properties and in vitro behavior of nanofiber-hydrogel composites for tissue engineering applications. Nanotechnology, 2012. 23(9): p. 095705.
4. Tsoi, B., et al., Safety of tissue expander implant versus autologous abdominal tissue breast reconstruction in postmastectomy breast cancer patients: a systematic review and meta-analysis. Plastic and reconstructive surgery, 2014. 133(2): p. 234-249.
5. Powell, S. K., et al., Past, present, and future of soft-tissue prosthetics: advanced polymers and advanced manufacturing. Advanced Materials, 2020. 32(42): p. 2001122.
6. Bellas, E., et al., Injectable silk foams for soft tissue regeneration. Advanced healthcare materials, 2015. 4(3): p. 452-459.
7. Pachence, J. M., Collagen-based devices for soft tissue repair. Journal of biomedical materials research, 1996. 33(1): p. 35-40.
8. Young, D. A., et al., Injectable hydrogel scaffold from decellularized human lipoaspirate. Acta biomaterialia, 2011. 7(3): p. 1040-1049.
9. Varma, D. M., et al., Injectable carboxymethylcellulose hydrogels for soft tissue filler applications. Acta Biomaterialia, 2014. 10(12): p. 4996-5004.
10. Khetan, S., et al., Degradation-mediated cellular traction directs stem cell fate in covalently crosslinked three-dimensional hydrogels. Nature materials, 2013. 12(5): p. 458-465.
11. Atashroo, D., et al., Studies in fat grafting: Part II. Effects of injection mechanics on material properties of fat. Plastic and reconstructive surgery, 2014. 134(1): p. 39.
12. Desai, R. M., et al., Versatile click alginate hydrogels crosslinked via tetrazine-norbornene chemistry. Biomaterials, 2015. 50: p. 30-37.
13. Tezel, A. and G. H. Fredrickson, The science of hyaluronic acid dermal fillers. Journal of Cosmetic and Laser Therapy, 2008. 10(1): p. 35-42.
14. Wang, F., et al., In vivo stimulation of de novo collagen production caused by cross-linked hyaluronic acid dermal filler injections in photodamaged human skin. Archives of dermatology, 2007. 143(2): p. 155-163.
15. Sussman, E. M., et al., Porous implants modulate healing and induce shifts in local macrophage polarization in the foreign body reaction. Annals of biomedical engineering, 2014. 42(7): p. 1508-1516.
16. Brown, B. N., et al., Macrophage phenotype as a predictor of constructive remodeling following the implantation of biologically derived surgical mesh materials. Acta biomaterialia, 2012. 8(3): p. 978-987.
17. Coburn, J., et al., Biomimetics of the extracellular matrix: an integrated three-dimensional fiber-hydrogel composite for cartilage tissue engineering. Smart structures and systems, 2011. 7(3): p. 213.
18. Zhou, Z., et al., Nanofiber-reinforced decellularized amniotic membrane improves limbal stem cell transplantation in a rabbit model of corneal epithelial defect. Acta biomaterialia, 2019. 97: p. 310-320.
19. Li, X., et al., Nanofiber-hydrogel composite-mediated angiogenesis for soft tissue reconstruction. Science translational medicine, 2019. 11(490): p. eaau6210.
20. Li, X., et al., The effect of a nanofiber-hydrogel composite on neural tissue repair and regeneration in the contused spinal cord. Biomaterials, 2020. 245: p. 119978.
21. Ibrahim, S., Q. K. Kang, and A. Ramamurthi, The impact of hyaluronic acid oligomer content on physical, mechanical, and biologic properties of divinyl sulfone-crosslinked hyaluronic acid hydrogels. Journal of Biomedical Materials Research Part A, 2010. 94(2): p. 355-370.
22. Haridas, N. and M. Rosemary, Effect of steam sterilization and biocompatibility studies of hyaluronic acid hydrogel for viscosupplementation. Polymer degradation and stability, 2019. 163: p. 220-227.
23. Lei, Y., et al., The spreading, migration and proliferation of mouse mesenchymal stem cells cultured inside hyaluronic acid hydrogels. Biomaterials, 2011. 32(1): p. 39-47.
24. Amici, S. A., et al., CD38 is robustly induced in human macrophages and monocytes in inflammatory conditions. Frontiers in immunology, 2018. 9: p. 1593.
25. Bund, T., et al., Analysis of chronic inflammatory lesions of the colon for BMMF Rep antigen expression and CD68 macrophage interactions. Proceedings of the National Academy of Sciences, 2021. 118(12).
26. Alvarado-Vazquez, P. A., et al., Macrophage-specific nanotechnology-driven CD163 overexpression in human macrophages results in an M2 phenotype under inflammatory conditions. Immunobiology, 2017. 222(8-9): p. 900-912.
27. Duijvestijn, A. M., et al., Antibodies defining rat endothelial cel/s: RECA-1, a pan-endothelial cell-specific monoclonal antibody. Lab Invest, 1992. 66(4): p. 459-466.
28. Kelly-Goss, M. R., et al., Targeting pericytes for angiogenic therapies. Microcirculation, 2014. 21(4): p. 345-357.
29. Hansen, J. S., et al., Visualization of lipid directed dynamics of perilipin 1 in human primary adipocytes. Scientific reports, 2017. 7(1): p. 1-14.
30. Martella, E., et al., Secreted adiponectin as a marker to evaluate in vitro the adipogenic differentiation of human mesenchymal stromal cells. Cytotherapy, 2014. 16(11): p. 1476-1485.
31. Xu, J., et al., Lysosomal protein surface expression discriminates fat-from bone-forming human mesenchymal precursor cells. Elife, 2020. 9: p. e58990.
32. Craig, D. J., et al., Blood Vessel Resident Human Stem Cells in Health and Disease. Stem Cells Translational Medicine, 2022.
33. Seidlits, S. K., et al., The effects of hyaluronic acid hydrogels with tunable mechanical properties on neural progenitor cell differentiation. Biomaterials, 2010. 31(14): p. 3930-3940.
34. Baier Leach, J., et al., Photocrosslinked hyaluronic acid hydrogels: natural, biodegradable tissue engineering scaffolds. Biotechnology and bioengineering, 2003. 82(5): p. 578-589.
35. Guimaraes, C. F., et al., The stiffness of living tissues and its implications for tissue engineering. Nature Reviews Materials, 2020. 5(5): p. 351-370.
36. Peattie, R., et al., Stimulation of in vivo angiogenesis by cytokine-loaded hyaluronic acid hydrogel implants. Biomaterials, 2004. 25(14): p. 2789-2798.
37. Desmoulière, A., I. A. Darby, and G. Gabbiani, Normal and pathologic soft tissue remodeling: role of the myofibroblast, with special emphasis on liver and kidney fibrosis. Laboratory investigation, 2003. 83(12): p. 1689-1707.
38. Latorre, M. and J. D. Humphrey, Critical roles of time-scales in soft tissue growth and remodeling. APL bioengineering, 2018. 2(2): p. 026108.
39. Zöller, N., et al., Collagen I promotes adipocytogenesis in adipose-derived stem cells in vitro. Cells, 2019. 8(4): p. 302.
40. Khunmanee, S., Y. Jeong, and H. Park, Crosslinking method of hyaluronic-based hydrogel for biomedical applications. Journal of tissue engineering, 2017. 8: p. 2041731417726464.

### EQUIVALENTS

It is understood that the detailed examples and embodiments described herein are given by way of example for illustrative purposes only, and are in no way considered to be limiting to the invention. Various modifications or changes in light thereof will be suggested to persons skilled in the art and are included within the spirit and purview of this application and are considered within the scope of the appended claims. For example, the relative quantities of the ingredients may be varied to optimize the desired effects, additional ingredients may be added, and/or similar ingredients may be substituted for one or more of the ingredients described. Additional advantageous features and functionalities associated with the systems, methods, and processes will be apparent from the appended claims. Moreover, those skilled in the art will recognize, or be able to ascertain using no more than routine experimentation, many equivalents to the specific embodiments of the invention described herein. Such equivalents are intended to be encompassed by the following claims.

1. A fiber-hydrogel composition for administration to a subject, the composite comprising:
fibers comprising one or more collagen materials; and
one or more hydrogel materials;
wherein fibers and one or more hydrogel materials are covalently bonded.

2. The composition of claim 1, wherein the fibers comprise one or more selected from collagen, gelatin, cellulose, modified cellulose, cellulose acetate, HPMC, ethyl cellulose, silk, chitosan, keratin, elastin, elastin-like polypeptides, tropoelastin, and hyaluronic acid.

3. The composition of claim 1 or 2 wherein the fibers are nanofibers or microfibers.

4. The composition of any one of claims 1 through 3 wherein the fibers comprise one or more from bovine type I collagen or gelatin, and its derivatives.

5. The composition of any one of claims 1 through 4 wherein the fibers comprise one or more recombinant collagen materials.

6. The composition of any one of claims 1 through 5 wherein the fibers comprise one or more recombinant human collagen materials.

7. The composition of any one of claims 1 through 6 wherein the collagen fiber comprises a type I bovine collagen nanofiber or fragments thereof.

8. The composition of any one of claims 1 through 7, wherein the collagen fiber is electrospun or centrifugal spun.

9. The composition of any one of claims 1 through 8 wherein the one or more hydrogel materials comprise one or more of hyaluronic acid (HA), collagen, chitosan, alginate, polyvinyl acetate (PVA), gelatin, polyethylene glycol (PEG) and other glycol ethers, chondroitin sulfate or a cellulose material.

10. The composition of any one of claims 1 through 8 wherein the one or more hydrogel materials comprise one or more hyaluronic acid (HA) materials.

11. The composition of any one of claims 1 through 10 wherein the one or more hydrogel materials are covalently bonded to the type I bovine collagen nanofiber sheet or fragments thereof.

12. The composition of any one of claims 1 through 11 wherein the collagen nanofiber is retained inside the fiber-hydrogel composite.

13. The composition of any one of claims 1 through 12 wherein the composition further comprises a crosslinking agent or reacted form of a crosslinking agent.

14. The composition of claim 13 wherein the crosslinking agent generates interfacial bonding between the collagen nanofiber and the HA chain.

15. The composition of claim 13 or 14 wherein the crosslinking agent comprises one or more selected from difunctional epoxide-based crosslinkers, 1,4-butanediol diglycidyl ether (BDDE), divinyl sulfone (DVS), PEG molecules with terminal epoxide functional groups, or HA-reactive agents.

16. The composition of claim 13 or 14 wherein the crosslinking agent comprises DVS or BDDE.

17. The composition of claim 16 wherein a concentration of the DVS ranges from about 0.5 v/v % to about 2.5 v/v % based on the total volume of the fiber-hydrogel composite.

18. The composition of any one of claims 1 through 17 wherein a concentration of the HA ranges from about 0.5 w/v % to about 2 w/v % based on the total volume of the fiber-hydrogel composite.

19. The composition of any one of claims 1 through 18 wherein a fiber loading density of the collagen nanofiber ranges from about 1 w/v % to about 3 w/v % based on the total volume of the fiber-hydrogel composite.

20. The composition of any one of claims 1 through 19, wherein a concentration of the fibers ranges from about 0.1 w/v % to about 20 w/v %, a concentration of the hydrogel material ranges from about 0.5 w/v % to about 10 w/v %, and a concentration of a crosslinking agent ranges from about 0.05 v/v % to about 5 v/v %, based on the total volume of the composition.

21. The composition of claim 20, wherein the concentration of the fibers ranges from about 1.5 w/v % to about 3.0 w/v %, the concentration of the hydrogel material ranges from about 0.8 w/v % to about 2 w/v %, and a concentration of a crosslinking agent ranges from about 0.5 v/v % to about 2.5 v/v %, based on the total volume of the composition.

22. The composition of any one of claims 1 to 21, wherein a pH of the composition ranges from about 7.0 to about 7.4 in an isotonic solution.

23. The composition of any one of claims 1 to 22, wherein the composition is formulated in a form of a sheet or an injectable fluid.

24. The composition of any one of claims 1 to 23, wherein the composition comprises one or more porous structures.

25. The composition of any one of claims 1 to 24, wherein a storage modulus of the composition ranges from about 100 to about 2 kPa.

26. The composition of claim 25, wherein a storage modulus of the composition ranges from about 100 to about 500 Pa.

27. The composition of any one of claims 1 to 26, wherein the composition is formed in a microbead having a mean diameter in a range of about 50 μm to about 1000 μm.

28. A soft tissue device comprising a composition of any one of claims 1 to 27.

29. An implant for promoting angiogenesis, comprising a composition of any one of claims 1 to 27.

30. An implant for adipose tissue formation, comprising a composition of any one of claims 1 to 27.

31. An implant for vasculature formation, comprising a composition of any one of claims 1 to 27.

32. A kit comprising
a composition of any one of claims 1 to 27; and
an applicator.

33. The kit of claim 32, wherein the applicator is an injector with a needle or cannulas.

34. The kit of claim 32 or 33, further comprising a vial comprising one or more of a salt, a buffer solution, and therapeutic agent.

35. A method of producing a fiber-hydrogel composite comprising:
contacting a crosslinking agent with fibers comprising one or more collagen materials and hyaluronic acid (HA) to obtain a fiber-hydrogel composite,
wherein the fibers are nanofibers or microfibers, and
wherein the HA is bonded to the fibers by the crosslinking agent to form a composite network.

36. The method of claim 35, further comprising modulating crosslinking condition.

37. The method of claim 35, wherein the crosslinking condition is under a basic condition.

38. The method of claim 37, wherein a pH of the crosslinking condition ranges from about 10 to about 14.

39. The method of any one of claims 35 to 38, wherein the contacting is performed for about 30 minutes to about 4 hours.

40. The method of any one of claims 35 to 39, wherein the contacting is performed at a temperature of about 20 to 90 degrees C.

41. The method of claim 40, wherein the contacting is performed at a temperature of about 37 degrees C.

42. The method of any one of claims 35 to 41, further comprising processing the fiber-hydrogel composite to particularize into to microbeads.

43. The method of claim 42, wherein a mean diameter of the microbeads ranges from about 50 μm to about 1,000 μm.

44. The method of any one of claims 35 to 43, wherein the processing comprises mechanical milling or mechanical screening by applying shear using a mesh.

45. The method of any one of claims 35 to 44, further comprising sterilizing the fiber-hydrogel composite.

46. The method of any of the claims 35 to 45, wherein the fibers comprise one or more from collagen, chitosan, alginate, gelatin, and derivatives.

47. The method of claim 46, wherein the fibers comprise a collagen nanofiber.

48. The method of claim 47, wherein the collagen nanofiber comprises a type I bovine collagen nanofiber or fragments thereof or a human collagen material.

49. The method of any one of claims 35 to 48, wherein the collagen nanofiber is electrospun and/or cryomilled.

50. The method of claim 41, wherein the HA is covalently bonded to the collagen nanofiber.

51. The method of any one of claims 35 to 50, wherein the crosslinking agent generates interfacial bonding between the collagen nanofiber and the HA.

52. The methods of any one of claims 35 to 51, wherein the crosslinking agent comprises one or more selected from difunctional epoxide-based crosslinkers, 1,4-butanediol diglycidyl ether (BDDE), divinyl sulfone (DVS), PEG molecules with terminal epoxide functional groups, and HA-reactive agents.

53. The method of any one of claims 35 to 52, wherein the crosslinking agent comprises DVS.

54. The method of claim 53, wherein a concentration of the DVS ranges from about 0.5 v/v % to about 2.5 v/v % based on the total volume of the fiber-hydrogel composite.

55. The method of any one of claims 35 to 54, wherein a concentration of the HA ranges from about 0.5 w/v % to about 2 w/v % based on the total volume of the fiber-hydrogel composite.

56. The method of any one of claims 35 to 55, a fiber loading density of the collagen nanofiber ranges from about 1 w/v % to about 3 w/v % based on the total volume of the fiber-hydrogel composite.

57. The method of any one of claims 35 to 56, wherein a concentration of the collagen fibers from about 0.1 w/v % to about 20 w/v %, a concentration of the HA ranges from about 0.5 w/v % to about 10 w/v %, and a concentration of the crosslinking agent ranges from about 0.05 v/v % to about 5 v/v %, based on the total volume of the fiber-hydrogel composite.

58. The method of any one of claims 35 to 56, wherein the concentration of the collagen fibers ranges from about 1.5 w/v % to about 3.0 w/v %, the concentration of the HA ranges from about 0.8 w/v % to about 2 w/v %, and a concentration of the crosslinking agent ranges from about 0.5 v/v % to about 2.5 v/v %, based on the total volume of the fiber-hydrogel composite.

59. The method of any one of claims 35 to 58, wherein a pH of the fiber-hydrogel composite ranges from about 7.0 to about 7.4 in an isotonic solution.

60. The method of any one of claims 35 to 59, further comprising fabricating the fiber-hydrogel composite in a sheet form or an injectable bead form.

61. The method of any one of claims 35 to 60, wherein the fiber-hydrogel composite comprises one or more porous structures.

62. The method of any one of claims 35 to 61, wherein a storage modulus of the fiber-hydrogel composite ranges from about 100 to about 500 Pa.

63. A method of forming adipose tissue in a subject comprising administering a composition of any one of claims 1 to 27 to the subject.

64. The method of claim 63, wherein prolonged in vivo retention of composition or enhanced host cell infiltration is induced by the composition, optionally in the absence of incorporation of cells or growth factors.

65. The method of claim 64, wherein the subject exhibits neo-vasculature formation facilitated by M2 macrophage polarization.

66. A method of delivering a cell or tissue in a subject, comprising:
encapsulating one or more cells or tissues in a composition of any one of claims 1 to 27 to form a mixture; and
applying the mixture to a target site in the subject.

67. A method of claim 66, wherein the tissue is a processed adipose tissue.

68. The method of claim 66, wherein the cell is one or more types of cells selected from a group consisting of adipose-derived stem cells, adipocytes, or combinations thereof.

69. A method of delivering a pharmaceutical agent in a subject, comprising:
combining a pharmaceutical agent and a composition of any one of claims 1 to 27 to form a mixture; and
applying the mixture to an intended site of delivery.

70. An implant device a fiber-hydrogel composite of any one of claims 1 to 27.
